(19) United States
(12) Patent Application Publication
(10) Pub. No.: US 2024/0349744 A1
(43) Pub. Date: Oct. 24, 2024

(54) COMPOSITIONS AND METHODS FOR PRODUCING FERMENTED PLANT-BASED COMPOSITIONS HAVING CREAM FLAVOR

(71) Applicant: INTERNATION N&H DENMARK APS, Kongens Lyngby (DK)

(72) Inventor: Elise Manoury, Chatellerault (FR)

(21) Appl. No.: 18/580,928

(22) PCT Filed: Jul. 28, 2022
(86) PCT No.: PCT/EP2022/071210

§ 371 Date: Jan. 19, 2024

Related U.S. Application Data
(60) Provisional application No. 63/226,957, filed on Jul. 29, 2021.

(30) Foreign Application Priority Data
Nov. 4, 2021 (EP) 21206401.8

(51) Int. Cl. A23C 11/10; A23L 11/30; A23L 11/50; C12N 1/20; C12R 1/225; C12R 1/46

(52) U.S. Cl. CPC A23C 11/106; A23L 11/37; A23L 11/50; C12N 1/205; A23V 2400/175; A23V 2400/249; C12R 2001/225; C12R 2001/46

(57) ABSTRACT

Provided herein are compositions and methods for producing fermented plant-based compositions, e.g., fermented dairy alternative food products, having cream flavor. The compositions and methods provided herein include bacteria, e.g., Lacticaseibacillus rhamnosus, useful for producing a ratio of acetaldehyde content to diacetyl content that produces a cream flavor in fermented plant-based compositions. In some embodiments, the bacteria, e.g., Lacticaseibacillus rhamnosus, decreases acetaldehyde content and/or increases diacetyl content in the fermented plant-based composition. Fermented plant-based compositions, including stored fermented plant-based compositions, produced according to the methods described herein are provided. Also provided are methods for identifying bacteria useful for producing fermented plant-based compositions having cream flavor.

### FIELD OF THE INVENTION

Provided herein are compositions and methods for producing fermented plant-based compositions, e.g., fermented dairy alternative food products, having cream flavor. The compositions and methods provided herein include bacteria, e.g., Lacticaseibacillus rhamnosus, useful for producing a ratio of acetaldehyde content to diacetyl content that produces a cream flavor in fermented plant-based compositions. In some embodiments, the bacteria, e.g., Lacticaseibacillus rhamnosus, decreases acetaldehyde content and/or increases diacetyl content in the fermented plant-based composition. Fermented plant-based compositions, including stored fermented plant-based compositions, produced according to the methods described herein are provided. Also provided are methods for identifying bacteria useful for producing fermented plant-based compositions having cream flavor.

### BACKGROUND TO THE INVENTION

Flavor perception is a complex sensory phenomenon that contributes to the acceptance and desirability of food products by consumers. In fermented plant-based dairy alternative products, such as plant-based yogurt alternatives, it is preferable to achieve organoleptic properties similar to those of fermented dairy products and minimize plant-based notes that are commonly identified as off-flavors. Cream flavor in particular can be an important flavor signature for consumer acceptance of fermented plant-based dairy alternatives.

Achieving a dairy-like sensory profile in fermented plant-based dairy alternatives has remained a challenge in the industry, and with a growing interest in plant-based diets and natural ingredients, consumer demand has lent an urgency to identifying solutions that do not rely on artificial means.

Thus, there exists a need for compositions and methods for naturally producing and improving cream flavor in fermented plant-based compositions, such as fermented plant-based dairy alternatives. The compositions and methods provided herein address such needs.

### SUMMARY OF THE INVENTION

In aspects are provided methods for producing a fermented plant-based composition having a cream flavor, the methods including decreasing acetaldehyde content in a fermented plant-based composition. Also provided are methods for producing stored fermented plant-based compositions having a cream flavor, the methods including: (a) storing a fermented plant-based composition to produce a stored fermented plant-based composition; and (b) decreasing acetaldehyde content in the fermented plant-based composition during storage. In some embodiments, the methods include producing the fermented plant-based composition, the producing including fermenting a vegetal base inoculated with a starter culture to produce the fermented plant-based composition.

In aspects are provided methods for producing a fermented plant-based composition having a cream flavor, the methods including: (a) fermenting a vegetal base inoculated with a starter culture to produce a fermented plant-based composition; and (b) decreasing acetaldehyde content in the fermented plant-based composition.

In some embodiments, the methods provided herein further include increasing diacetyl content in the fermented plant-based composition. In some embodiments, the vegetal base comprises soy plant matter. In some embodiments, the soy plant matter is contained in an aqueous suspension. In some embodiments, the methods include storing the fermented plant-based composition to produce a stored fermented plant-based composition. In some embodiments, the acetaldehyde content is decreased in the fermented plant-based composition during storage. In some embodiments, the methods include increasing diacetyl content in the fermented plant-based composition during storage. In some embodiments, the acetaldehyde content is decreased to produce a ratio of acetaldehyde to diacetyl that is less than 0.7, 0.6, 0.5, 0.4, 0.3, 0.2, 0.1 or is 0. In some embodiments, the acetaldehyde content is decreased and the diacetyl content is increased to produce a ratio of acetaldehyde to diacetyl that is less than 0.7, 0.6, 0.5, 0.4, 0.3, 0.2, 0.1 or is 0.

In some embodiments, the starter culture contains one or more lactic acid bacteria. In some embodiments, the starter culture contains at least two lactic acid bacteria. In some embodiments, the starter culture contains bacteria from the genus Lactococcus, Lactobacillus, Streptococcus, Lacticaseibacillus, Leuconostoc, Pediococcus, or Bifidobacterium, or any combination of the foregoing. In some embodiments, the starter culture contains one or more Lacticaseibacillus rhamnosus strains. In some embodiments, the one or more of the Lacticaseibacillus rhamnosus strains is of vegetal origin. In some embodiments, the one or more Lacticaseibacillus rhamnosus strains are or include: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; or (c) the Lacticaseibacillus rhamnosus strain deposited under accession number DSM33850 at the DSMZ or a mutant thereof.

In some embodiments, the starter culture contains one or more Streptococcus thermophilus strains. In some embodiments, the one or more Streptococcus thermophilus strains are or include the Streptococcus thermophilus strain deposited under accession number DSM33651 at the DSMZ or a mutant thereof.

In some embodiments, the starter culture is or includes: (a) a Lacticaseibacillus rhamnosus strain deposited under accession number DSM33650 at the DSMZ or a mutant thereof and a Streptococcus thermophilus strain deposited under accession number DSM33651 at the DSMZ or a mutant thereof; (b) a Lacticaseibacillus rhamnosus strain deposited under accession number DSM22876 at the DSMZ or a mutant thereof and a Streptococcus thermophilus strain deposited under accession number DSM33651 at the DSMZ or a mutant thereof; or (c) a Lacticaseibacillus rhamnosus strain deposited under accession number DSM33850 at the DSMZ or a mutant thereof and a Streptococcus thermophilus strain deposited under accession number DSM33651 at the DSMZ or a mutant thereof.

In some embodiments, the starter culture contains one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus lactis subsp lactis strain, or a Lactococcus cremoris subsp cremoris strain.

In some embodiments, the inoculated vegetal base is fermented at a temperature above about 33° C., 35° C., 36° C., 37° C., 38° C., 39° C., 40° C., 41° C., 42° C., 43° C., 44° C., 45° C., or 50° C. In some embodiments, the fermented plant-based composition is stored at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or about 26° C. In some embodiments, the fermented plant-based composition is stored for: a) at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60 70, 80, 90, 100, 110, or 120 days; or b) at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60 70, 80, 90, 100, 110, or 120 days. In some embodiments, the fermented plant-based composition is stored for about 28 days at about 6° C. In some embodiments, the fermented plant-based composition is stored for about 14 days at about 6° C. In some embodiments, the fermented plant-based composition is stored for about 7 days at about 6° C.

In some embodiments, the cream flavor includes a cream aroma and/or cream odor. In some embodiments, the cream flavor is an increased cream flavor compared to a cream flavor of a fermented plant-based composition having an acetaldehyde content and a diacetyl content resulting in a ratio of acetaldehyde to diacetyl of equal to or greater than 0.7. In some embodiments, the increased cream flavor includes an increased cream aroma and/or cream odor compared to a cream aroma and/or cream odor of a fermented plant-based composition having an acetaldehyde content and a diacetyl content resulting in a ratio of acetaldehyde to diacetyl of equal to or greater than 0.7.

In aspects are provided compositions including a Lacticaseibacillus rhamnosus strain, wherein the Lacticaseibacillus rhamnosus strain decreases acetaldehyde in a fermented plant-based composition to produce a cream flavor. In some embodiments, the Lacticaseibacillus rhamnosus strain is of vegetal origin. In some embodiments, the Lacticaseibacillus rhamnosus strain is: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; or (c) the Lacticaseibacillus rhamnosus strain deposited under accession number DSM33850 at the DSMZ or a mutant thereof. In some embodiments, the composition further includes a Streptococcus thermophilus strain. In some embodiments, the Streptococcus thermophilus strain is the Streptococcus thermophilus strain deposited under accession number DSM33651 at the DSMZ or a mutant thereof. In some embodiments, the composition is or includes: (a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof; (b) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; or (c) the Lacticaseibacillus rhamnosus strain deposited under accession number DSM33850 at the DSMZ or a mutant thereof and the Streptococcus thermophilus strain deposited under accession number DSM33651 at the DSMZ or a mutant thereof. In some embodiments, the composition further includes one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus lactis subsp lactis strain, or Lactococcus cremoris subsp cremoris strain. In some embodiments, the composition is a starter culture. In some embodiments, the composition is a fermented plant-based food product.

In aspects is provided use of a Lacticaseibacillus rhamnosus to decrease acetaldehyde content in a fermented plant-based composition to produce cream flavor. In some embodiments, the Lacticaseibacillus rhamnosus increases diacetyl content in the fermented plant-based composition. In some embodiments, the Lacticaseibacillus rhamnosus strain is: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; or (c) the Lacticaseibacillus rhamnosus strain deposited under accession number DSM33850 at the DSMZ or a mutant thereof. In some embodiments, the acetaldehyde content is decreased to produce a ratio of acetaldehyde to diacetyl that is less than 0.7, 0.6, 0.5, 0.4, 0.3, 0.2, 0.1 or is 0. In some embodiments, the acetaldehyde content is decreased and the diacetyl content is increased to produce a ratio of acetaldehyde to diacetyl that is less than 0.7, 0.6, 0.5, 0.4, 0.3, 0.2, 0.1 or is 0. In some embodiments, the cream flavor comprises a cream aroma and/or cream odor. In some embodiments, the cream flavor is an increased cream flavor compared to a cream flavor of a fermented plant-based composition having an acetaldehyde content and a diacetyl content resulting in a ratio of acetaldehyde to diacetyl of equal to or greater than 0.7. In some embodiments, the increased cream flavor includes an increased cream aroma and/or cream odor compared to a cream aroma and/or cream odor of a fermented plant-based composition having an acetaldehyde content and a diacetyl content resulting in a ratio of acetaldehyde to diacetyl of equal to or greater than 0.7.

In aspects is provided a fermented plant-based composition obtained according to the methods or use provided herein. In some embodiments, the fermented plant-based composition is a fermented plant-based food product. In some embodiments, the fermented plant-based composition is a fermented plant-based dairy alternative food product. In some embodiments, the wherein the fermented plant-based composition is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.

In aspects is provided a fermented plant-based food product having a cream flavor, the fermented plant-based food product containing a ratio of acetaldehyde content to diacetyl content that is less than 0.7, 0.6, 0.5, 0.4, 0.3, 0.2, 0.1 or is 0. In some embodiments, the fermented plant-based food product contains a Lacticaseibacillus rhamnosus strain that decreases acetaldehyde in the fermented plant-based food product. In some embodiments, the Lacticaseibacillus rhamnosus strain increases diacetyl in the fermented plant-based food product.

In some embodiments, the fermented plant-based food product contains: (a) a Lacticaseibacillus rhamnosus strain deposited under accession number DSM33650 or a mutant thereof; (b) a Lacticaseibacillus rhamnosus strain deposited under accession number DSM22876 or a mutant thereof; and/or (c) a Lacticaseibacillus rhamnosus strain deposited under accession number DSM33850 or a mutant thereof. In some embodiments, the fermented plant-based food product further contains a Streptococcus thermophilus strain. In some embodiments, the Streptococcus thermophilus strain is the Streptococcus thermophilus strain deposited under accession number DSM33651 or a mutant thereof. In some embodiments, the fermented plant-based food product is a fermented plant-based dairy alternative food product. In some embodiments, the fermented plant-based food product is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.

In aspects is provided a method of identifying bacteria capable of producing a fermented plant-based composition having cream flavor, the method including quantifying acetaldehyde content and diacetyl content in a fermented plant-based composition fermented in the presence of one or more bacteria. In some embodiments, the fermented plant-based composition is stored and the acetaldehyde content and diacetyl content are quantified during storage. In some embodiments, if the quantified acetaldehyde content and diacetyl content produce a ratio of acetaldehyde content to diacetyl content that is less than 0.7, 0.6, 0.5, 0.4, 0.3, 0.2, 0.1 or is 0, the one or more bacteria are identified as able to produce a fermented plant-based composition having cream flavor. In some embodiments, the one or more bacteria include a Lacticaseibacillus rhamnosus strain. In some embodiments, the Lacticaseibacillus rhamnosus is of vegetal origin. In some embodiments, the one or more bacteria include a Streptococcus thermophilus strain.

In some embodiments, the ratio of acetaldehyde to diacetyl described herein associated with cream flavor is reached after 28 days or less in storage. In some embodiments, the ratio of acetaldehyde to diacetyl described herein associated with cream flavor is reached after 21 days or less in storage. In some embodiments, the ratio of acetaldehyde to diacetyl described herein associated with cream flavor is reached after 14 days or less in storage. In some embodiments, the ratio of acetaldehyde to diacetyl described herein associated with cream flavor is reached after 7 days or less in storage.

In aspects is provided a composition including one or more bacteria identified as capable of producing a fermented plant-based composition having cream flavor according to the methods provided herein. In some embodiments, the composition is a starter culture or a fermented plant-based food product. In some embodiments, the fermented plant-based food product is a fermented plant-based dairy alternative food product.

Also provided herein are kits containing a composition described herein and instructions for use.

In aspects is provided a Lacticaseibacillus rhamnosus strain deposited under accession number DSM33850 or a mutant thereof. In aspects is provided a Lacticaseibacillus rhamnosus strain deposited under accession number DSM22876 or a mutant thereof. In aspects is provided a Streptococcus thermophilus strain deposited under accession number DSM33651 at the DSMZ or a mutant thereof.

Each of the aspects and embodiments described herein are capable of being used together, unless excluded either explicitly or clearly from the context of the embodiment or aspect.

### BRIEF DESCRIPTION OF THE FIGURES

FIG. 1 shows a scatterplot of 2,3-butanedione (diacetyl) and acetaldehyde content (in ppm) after 1, 7, 14, 21, and 28 days in storage at 6° C. for five soy-based yogurt alternatives produced by fermentation with an exemplary Streptococcus thermophilus strain (DSM33651) alone or in combination with an exemplary L. rhamnosus strain (DSM24616, DSM33650, DSM22876, or DSM33850) as indicated. The 2,3-butanedione and acetaldehyde content (in ppm) is also shown for the soy substrate prior to fermentation.

FIG. 2 shows a scatterplot of 2,3-butanedione (diacetyl) and acetaldehyde content (in ppm) after 7 days of storage at 6° C. for soy-based yogurt alternatives produced by fermentation with one of two industrial starter cultures (Industrial starter 1 or Industrial starter 2) alone or in combination with exemplary L. rhamnosus strain DSM33650.

FIG. 3 shows the acetaldehyde and 2,3-Butanedione contents (in ppm, axis 1) in soy yogurt alternative and sensory cream/milk odour evaluation (axis 2). 1: yogurt alternative fermented with industrial starter culture alone. 2: yogurt alternative fermented with industrial starter culture in combination with L. rhamnosus strain DSM33650

### DETAILED DESCRIPTION OF THE INVENTION

Provided herein are compositions and methods for producing a fermented plant-based composition, e.g., a fermented plant-based food product, having a cream flavor. As described herein, it was surprisingly found that fermenting vegetal bases, e.g., soy, with bacteria described herein could produce levels of acetaldehyde and diacetyl (also referred to as 2,3-butanedione) that resulted in an increased cream flavor in fermented plant-based compositions.

While it has been appreciated that diacetyl provides characteristic flavors of butter and can assist in cream flavor perception, it was not previously contemplated that a diacetyl content greater than an acetaldehyde content would associate, e.g., correlate, with a cream flavor in a fermented plant-based food product, nor that such a cream flavor would be increased compared to a cream flavor in fermented plant-based food products having similar levels of acetaldehyde and diacetyl or an acetaldehyde content greater than the diacetyl content. Thus, the methods and compositions provided herein harness the surprising findings described herein (see, e.g., Examples) that fermented plant-based compositions having a higher level of diacetyl than acetaldehyde have a cream flavor that is increased compared to cream flavor in fermented plant-based compositions having about equal levels of acetaldehyde and diacetyl or an acetaldehyde content that is greater than a diacetyl content.

The use of bacterial strains described herein for achieving diacetyl and acetaldehyde levels associated with cream flavor described herein, offers a natural solution to increase cream flavor in fermented plant-based compositions. The demonstrated ability of the compositions and methods provided herein to increase cream flavor without the use of additives or artificial flavors may be desirable to certain consumers.

In some aspects, the methods and compositions described herein allow a relative content, e.g., ratio, of acetaldehyde to diacetyl correlated with cream flavor to be achieved in the fermented plant-based compositions by modulating the levels of acetaldehyde and diacetyl in the fermented plant-based compositions. In some embodiments, the methods and compositions described herein facilitate decreases in the level of acetaldehyde and/or increases in the level of diacetyl in the fermented plant-based compositions to achieve a relative content, e.g., ratio, of acetaldehyde to diacetyl correlated with cream flavor. In some embodiments, the cream flavor produced by the methods and compositions provided herein is increased compared to methods and compositions for producing cream flavor in fermented plant-based compositions that do not achieve the relative content, e.g., ratio, of acetaldehyde to diacetyl as described herein.

In some embodiments, the methods provided herein include the use of bacteria or bacterial compositions, e.g., starter cultures, containing Lacticaseibacillus rhamnosus. In some embodiments, the Lacticaseibacillus rhamnosus modulates (e.g., decreases) acetaldehyde content in a fermented plant-based composition. In some embodiments, the Lacticaseibacillus rhamnosus modulates independently (e.g., independently increases or decreases) acetaldehyde and diacetyl content in a fermented plant-based composition. In some embodiments, the Lacticaseibacillus rhamnosus decreases the acetaldehyde content in a fermented plant-based composition. In some embodiments, the Lacticaseibacillus rhamnosus increases the diacetyl content in a fermented plant-based composition. In some embodiments, the Lacticaseibacillus rhamnosus decreases the acetaldehyde content and increases the diacetyl content in a fermented plant-based composition. In some embodiments, the bacterial composition, e.g., starter culture, further includes Streptococcus thermophilus.

In some embodiments, the bacteria and bacterial compositions described herein, e.g., starter cultures, produce a diacetyl content in the fermented plant-based composition that is greater than the acetaldehyde content. In some embodiments, the bacteria and bacterial compositions described herein, e.g., starter cultures, produce an increased diacetyl content compared to the acetaldehyde content at a relative content, e.g., ratio, of acetaldehyde to diacetyl that is associated with a cream flavor, e.g., an increased cream flavor. In some cases, achieving a relative content, e.g., ratio, of acetaldehyde to diacetyl that associates with a cream flavor, e.g., an increased cream flavor, is accomplished by fermenting a vegetal base using a bacteria or bacterial composition, e.g., starter culture, described herein that modulates (e.g., increases, decreases) levels of acetaldehyde and/or diacetyl. In some embodiments, the bacteria and bacterial compositions described herein, e.g., starter cultures, decrease the level of acetaldehyde in the fermented plant-based composition. In some embodiments, the bacteria and bacterial compositions described herein, e.g., starter cultures, increase the level of diacetyl in the fermented plant-based composition. In some embodiments, the bacteria and bacterial compositions described herein, e.g., starter cultures, increase the level of diacetyl or decrease the level of acetaldehyde in the fermented plant-based composition. In some embodiments, the bacteria and bacterial compositions described herein, e.g., starter cultures, increase the level of diacetyl and decrease the level of acetaldehyde in the fermented plant-based composition.

In some embodiments, the content, e.g., relative content, of acetaldehyde to diacetyl is expressed as a ratio (e.g., as a quotient) of acetaldehyde content to diacetyl content. In some embodiments, the bacteria and bacterial compositions described herein, e.g., starter cultures, independently modulate (e.g., decrease or increase) the level of acetaldehyde and diacetyl to produce a ratio of acetaldehyde to diacetyl that is less than 1, e.g., less than about 0.9, 0.8, 0.7, 0.6, 0.5, 0.4, 0.3, 0.2, 0.1, 0.075, 0.05, 0.01, or is 0. In some cases, the content, e.g., relative content, of acetaldehyde to diacetyl is expressed as a percentage. For example, in some cases, the bacteria and bacterial compositions described herein, e.g., starter cultures, independently modulate (e.g., decrease or increase) the level of acetaldehyde and diacetyl to produce a percentage of acetaldehyde to diacetyl that is less than 100%, e.g., less than about 90%, 80%, 70%, 60%, 50%, 40%, 30%, 20%, 10%, 7.5%, 5%, 1%, or 0%. In some cases, the content, e.g., relative content, of acetaldehyde to diacetyl is expressed as a fold, e.g., 2-fold, 3-fold. Any means suitable for numerically expressing the relationship between the acetaldehyde content and the diacetyl content may be used.

In some cases, the cream flavor in the fermented plant-based compositions produced according to the methods and compositions provided herein is not immediately present, e.g., according to a sensory analysis, in the fermented plant-based compositions following fermentation. For example, in some cases, the acetaldehyde to diacetyl content that is correlated with a cream flavor, e.g., an increased cream flavor, in fermented plant-based compositions, e.g., a ratio of acetaldehyde to diacetyl that is less than 1, occurs over time, e.g., an amount of time after fermentation has stopped. As such, in some embodiments, the cream flavor in the fermented plant-based compositions emerges over time as changes in acetaldehyde content and/or diacetyl content evolve. Thus, in some embodiments, the methods provided herein include storing the fermented plant-based compositions, for example as described in Section I-D below. In some embodiments, the fermented plant-based composition is stored under refrigeration, for example as described in Section I-D below. In some embodiments, the level of acetaldehyde decreases over time in storage. In some embodiments, the level of diacetyl increases over time in storage. In some embodiments, the level of acetaldehyde decreases or the level of diacetyl increases over time in storage. In some embodiments, the level of acetaldehyde decreases and the level of diacetyl increases over time in storage.

In some embodiments, the cream flavor in the fermented plant-based composition develops during storage. In some embodiments, the cream flavor in the fermented plant-based composition increases during storage. For example, in some cases, an organoleptic profile indicative of cream flavor changes such that increases in cream aroma and/or cream odor occur with additional time in storage. In some embodiments, the cream flavor in the fermented plant-based composition develops during storage and then reaches a stable level of cream flavor. For example, in some cases, an organoleptic profile indicative of cream flavor remains stable, e.g., lacks appreciable (e.g., statistically significant, sensorily detectable) increases or decreases in cream aroma and/or cream odor with additional time in storage. Methods for producing and assessing organoleptic profiles include those described in Section I-E and, for example, as described in Example 3.

In some embodiments, a fermented plant-based composition having cream flavor is a fermented plant-based composition that has been stored for a duration of time. In some embodiments, the fermented plant-based composition having cream flavor is a stored fermented plant-based composition. In some embodiments, the duration of storage of the fermented plant-based composition is or is about the amount of time needed for the acetaldehyde to decrease, and optionally the level of diacetyl to increase, to levels, e.g., relative quantities, ratios, that correlate with cream flavor in the fermented plant-based composition, e.g., stored fermented plant-based composition.

In some embodiments, the cream flavor in the fermented plant-based compositions produced according to the methods and compositions provided herein is associated, e.g., correlated, with specific levels of diacetyl and/or acetaldehyde in the fermented plant-based compositions, and/or relative quantities and ratios thereof. See, e.g., Section I-E below. For example, in some embodiments, the cream flavor is associated with a level of diacetyl that is greater than the level of acetaldehyde. In some embodiments, the cream flavor is associated, e.g., correlated, with a level of acetaldehyde content and a level of diacetyl in the fermented plant-based compositions that results in a ratio of acetaldehyde to diacetyl that is less than 1, e.g., less than about 0.9, 0.85, 0.8, 0.75, 0.7, 0.6, 0.5, 0.4, 0.3, 0.2, 0.1, 0.075, 0.05, 0.025, or 0.01, or is 0. In some embodiments, the level of diacetyl in the fermented plant-based composition is at least 1 ppm, e.g., at least about 1.5 ppm, 2 ppm, 3 ppm, 4 ppm, 5 ppm, 10 ppm, 15 ppm, or 20 ppm. In some embodiments, the level of acetaldehyde in the fermented plant-based composition is less than 1 ppm, e.g., less than about 0.9 ppm, 0.8 ppm, 0.7 ppm, 0.6 ppm, 0.5 ppm, 0.4 ppm, 0.3 ppm, 0.2 ppm, 0.1 ppm, 0.05 ppm, 0.01 ppm, or below a lower limit of detection and/or quantification by instrumentation. In some embodiments, the lower limit of quantification is 0.125 ppm. In some embodiments, the level of diacetyl content in the fermented plant-based composition is at least 1 ppm, e.g., at least about 1.5 ppm, 2 ppm, 3 ppm, 4 ppm, 5 ppm, 10 ppm, 5 ppm, or 20 ppm and the level of acetaldehyde is such that the fermented plant-based composition has a ratio of acetaldehyde to diacetyl that is less than 1, e.g., less than about 0.9, 0.8, 0.7, 0.6, 0.5, 0.4, 0.3, 0.2, 0.1, 0.075, 0.05, 0.025, or 0.01, or is 0 (e.g., when the level of acetaldehyde is below a level of quantification or detection). In some embodiments, only the level of acetaldehyde is decreased to produce a ratio of acetaldehyde to diacetyl associated with cream flavor. In some embodiments, the cream flavor of the fermented plant-based composition is increased compared to a fermented plant-based composition having an acetaldehyde to diacetyl ratio of, of about, or of greater than 1. The terms level, content, and concentration may be used interchangeably herein to describe amounts of acetaldehyde, diacetyl, or other volatile organic compounds.

In some embodiments, the cream flavor of the fermented plant-based composition produced according to the methods and compositions provided herein includes or is a cream aroma. Cream aroma is perceived via the retronasal pathway, for example once the food product, e.g., fermented plant-based composition, is in the mouth. In some embodiments, the cream flavor of the fermented plant-based composition produced according to the methods and compositions provided herein includes or is a cream odor. Cream odor is perceived via the nose using the orthonasal pathway (smell). In some embodiments, the cream flavor of the fermented plant-based composition produced according to the methods and compositions provided herein includes or is a cream aroma or a cream odor. In some embodiments, the cream flavor of the fermented plant-based composition produced according to the methods and compositions provided herein includes or is a cream aroma and a cream odor.

In some embodiments, the cream flavor of the fermented plant-based composition produced according to the methods and compositions provided herein is an increased cream flavor. For example, the cream flavor of the fermented plant-based composition produced according to the methods and compositions provided herein may be increased compared to a fermented plant-based composition that does not have or is not produced such that levels of acetaldehyde and diacetyl, or relative quantities, e.g., ratios, thereof, that correlate with the cream flavor according to the compositions and methods provided herein are achieved. Thus, in some embodiments, the cream flavor of the fermented plant-based composition produced according to the methods and compositions provided herein includes or is an increased cream aroma. In some embodiments, the cream flavor of the fermented plant-based composition produced according to the methods and compositions provided herein includes or is an increased cream odor. In some embodiments, the cream flavor of the fermented plant-based composition produced according to the methods and compositions provided herein includes or is an increased cream aroma or an increased cream odor. In some embodiments, the cream flavor of the fermented plant-based composition produced according to the methods and compositions provided herein includes or is an increased cream aroma and an increased cream odor. In some embodiments, the increase in cream flavor, e.g., increased cream aroma and/or increased cream odor, is determined in comparison to a fermented plant-based composition that does not have or is not produced such that levels of acetaldehyde and diacetyl, or relative quantities, e.g., ratios, thereof, that correlate with the cream flavor according to the compositions and methods provided herein are achieved.

In some embodiments, the cream flavor, e.g., increased cream flavor, including cream aromas and/or cream odors thereof, are determined by a trained panel of sensory assessors.

Also provided herein are fermented plant-based compositions that are food products (e.g., fermented plant-based food products) produced according the methods provided herein, including embodiments thereof. In some embodiments, the fermented plant-based composition (e.g., fermented plant-based food product) is a fermented dairy alternative food or feed product. In some embodiments, the fermented plant-based composition (e.g., fermented plant-based food product) is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative. The term alternative is used herein, in some embodiments, to indicate that the food product is akin to a dairy food product but not produced using dairy substrates, e.g., dairy milk. In some embodiments, the fermented plant-based composition is a stored fermented plant-based composition (e.g., stored fermented plant-based food products).

Methods of identifying bacteria capable of producing a fermented plant-based composition, optionally a stored fermented plant-based composition, having cream flavor are also provided herein. In some embodiments, the method of identifying includes quantifying acetaldehyde and diacetyl content, and optionally changes thereof, in a fermented plant-based composition fermented in the presence of one or more bacteria. In some embodiments, the method of identifying includes determining the relative content, e.g., ratio, of acetaldehyde to diacetyl in a fermented plant-based composition fermented in the presence of one or more bacteria. In some embodiments, the method of identifying includes quantifying acetaldehyde content and diacetyl content, and optionally changes thereof, in a fermented plant-based composition fermented in the presence of one or more bacteria and stored following fermentation. For example, quantifying acetaldehyde content and diacetyl content, and optionally changes thereof, occurs during storage of the fermented plant-based compositions. In some embodiments, the method of identifying includes determining the relative content, e.g., ratio, of acetaldehyde to diacetyl in a fermented plant-based composition fermented in the presence of one or more bacteria and stored following fermentation. For example, determining the relative content, e.g., ratio, of acetaldehyde to diacetyl occurs during storage of the fermented plant-based compositions. In some embodiments, the method of identifying includes quantifying acetaldehyde content and diacetyl content, and optionally changes thereof, in a stored fermented plant-based composition fermented in the presence of one or more bacteria. In some embodiments, the method of identifying includes determining the relative content, e.g., ratio, of acetaldehyde to diacetyl in a stored fermented plant-based composition fermented in the presence of one or more bacteria. In some embodiments, the quantifying of content and/or determination of relative content, e.g., ratio, as described herein takes place at different time points over the duration of storage. In some embodiments, the quantifying of content and/or determination of relative content, e.g., ratio, as described herein takes place continuously over the duration of storage. In some embodiments, the storage is carried out as described in Section I-D below. In some embodiments, the one or more bacteria useful for achieving diacetyl and/or acetaldehyde contents, and relative quantities and ratios thereof, correlated with cream flavor in a fermented plant-based composition as described herein (see, e.g., Section I-E) are identified as capable of producing a fermented plant-based composition, optionally a stored fermented plant-based composition, having cream flavor. In some embodiments, the one or more identified bacteria are contained in a composition such as a starter culture.

The compositions and methods provided herein are useful for naturally producing fermented plant-based compositions, e.g., fermented plant-based food products, having cream flavor, e.g., increased cream flavor, without the need for potentially undesirable and/or costly additives. Through the use of bacteria and bacterial compositions for fermentation, as described herein, the methods provided herein circumvent the need for additional processing steps, e.g., introducing additives to the composition, thus allowing production of the fermented plant-based composition to proceed without added time delays. The methods and compositions provided herein for producing fermented plant-based compositions with cream flavor can facilitate increased consumption of plant-based products, which may promote nutritional and health benefits, and may have a positive impact on the environment, for example when consumed as alternatives to dairy products. The methods and compositions described herein thus provide numerous advantages while also satisfying a consumer desire for fermented plant-based food products having a naturally increased cream flavor.

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise. For example, “a” or “an” include “at least one” or “one or more.”

The terms “comprising”, “comprises” and “comprised of” as used herein are synonymous with “including”, “includes” or “containing”, “contains”, and grammatical variants thereof, are inclusive or open-ended and do not exclude additional, non-recited members, elements or method steps. The terms “comprising”, “comprises” and “comprised of”, “including”, “includes” or “containing”, “contains”, and grammatical variants thereof also include the term “consisting of”.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Values and ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges may implicitly include the term “about” unless the context dictates otherwise.

All publications, including patent documents, scientific articles and databases, referred to in this application are incorporated by reference in their entirety for all purposes to the same extent as if each individual publication were individually incorporated by reference. Nothing herein is to be construed as an admission that such publications constitute prior art. If a definition set forth herein is contrary to or otherwise inconsistent with a definition set forth in the patents, applications, published applications and other publications that are herein incorporated by reference, the definition set forth herein prevails over the definition that is incorporated herein by reference.

### I. METHODS FOR PRODUCING FERMENTED PLANT-BASED COMPOSITIONS HAVING CREAM FLAVOR

The methods and compositions provided herein allow for the production of fermented plant-based compositions having cream flavor, where the cream flavor may be increased compared to fermented plant-based compositions produced according to different methods. In some embodiments, the cream flavor of the fermented plant-based composition produced according to the methods provided herein is increased compared to the cream flavor of fermented plant-based compositions produced according to other methods, for example, that do not control, e.g., modify, the relative amounts of acetaldehyde and diacetyl in the fermented plant-based compositions to levels associated, e.g., correlated, with cream flavor as described herein (see, e.g., Section I-E).

In some aspects, the methods and compositions provided herein achieve a fermented plant-based composition having relative quantities of acetaldehyde and diacetyl that are associated, e.g., correlated, with increased cream flavor. In some embodiments, the increased cream flavor has an increased cream aroma. In some embodiments, the increased cream flavor has an increased cream odor. In some embodiments, the increased cream flavor has an increased cream aroma or increased cream odor. In some embodiments, the increased cream flavor has an increased cream aroma and increased cream odor.

In some aspects, a method for producing a fermented plant-based composition having cream flavor is provided, the method including decreasing acetaldehyde content in a fermented plant-based composition. In some aspects, a method for producing a fermented plant-based composition having cream flavor is provided, the method including decreasing acetaldehyde content and increasing diacetyl content in a fermented plant-based composition.

In some aspects, a method for producing a fermented plant-based composition having cream flavor is provided, the method including fermenting a vegetal base inoculated with a starter culture to produce a fermented plant-based composition and decreasing acetaldehyde content in the fermented plant-based composition. In some aspects, a method for producing a fermented plant-based composition having cream flavor is provided, the method including fermenting a vegetal base inoculated with a starter culture to produce a fermented plant-based composition and decreasing acetaldehyde content and increasing diacetyl content in the fermented plant-based composition.

In some aspects, a method for producing a stored fermented plant-based composition having cream flavor is provided, the method including storing a fermented plant-based composition to produce the stored fermented plant-based composition and decreasing acetaldehyde content in the fermented plant-based compositions during storage. In some aspects, a method for producing a stored fermented plant-based composition having cream flavor is provided, the method including storing a fermented plant-based composition to produce the stored fermented plant-based composition and decreasing acetaldehyde content and increasing diacetyl content in the fermented plant-based compositions during storage.

In some aspects, a method of producing a cream flavor in a fermented plant-based composition during storage of the fermented plant-based compositions is provided, the method including decreasing acetaldehyde content in the fermented plant-based compositions during storage. In some aspects, a method of producing a cream flavor in a fermented plant-based composition during storage of the fermented plant-based compositions is provided, the method including decreasing acetaldehyde content and increasing diacetyl content in the fermented plant-based compositions during storage.

In some embodiments, the methods provided herein include producing a fermented plant-based composition. In some embodiments, the method for producing a fermented plant-based composition includes inoculating a vegetal base, for example as described in Section I-A below, with a bacteria or compositions thereof, for example as described in Section I-B below, and fermenting the inoculated vegetal base, for example as described in Section I-C below.

As indicated above, in some embodiments, the fermented plant-based composition is stored. A vegetal base that has undergone thermophilic fermentation and is subsequently stored may be referred to herein as a stored fermented plant-based composition. In some embodiments, the storage is under refrigeration. In some embodiments, the storage of the fermented plant-based composition, is carried out according to the methods described in Section I-D to produce the stored fermented plant-based composition.

In some embodiments, the relative quantities of acetaldehyde and diacetyl in the fermented plant-based composition that associate, e.g., correlate, with cream flavor, e.g., increased cream flavor, are achieved during storage of the fermented plant-based composition. In some embodiments, the levels, relative quantities, and ratios of acetaldehyde and diacetyl associated with cream flavor are described in Section I-E.

The methods and compositions provided herein are useful for producing a fermented plant-based composition that is a fermented plant-based food product having cream flavor, e.g., increased cream flavor. Exemplary fermented plant-based composition that are food products are described in Section I-F.

### A. Vegetal Base

In some embodiments, the fermented plant-based composition having cream flavor is produced by inoculating, e.g., with bacteria or a bacterial composition described herein, and fermenting a vegetal base. It is contemplated herein that a vegetal base for producing the fermented plant-based composition may be any raw and/or processed plant-based material, e.g., plant matter, that can be subjected to fermentation according to the methods provided herein.

In some embodiments, the vegetal base is or includes fully or partially hydrolysed plant matter, for example cereal. In some embodiments, the cereal is wheat, rye, spelt, barley, oat, millet, sorghum, rice, teff, or combinations thereof.

In some embodiments, the vegetal base is or includes plant matter from legumes. In some embodiments, the vegetal base is or includes pulses, for example split peas, field peas, dry peas, lentil, chickpeas, garbanzo bean, konda, navy bean, white navy bean, white pea bean, pea bean, cow pea, horse bean, haricot, pinot bean, mottled bean, small red bean, red Mexican bean, kidney bean, black bean, black turtle bean, cranberry bean, roman bean, speckled sugar bean, lima bean, haba bean, Madagascar bean, green gram, mung bean, green bean, black gram, urad dal, soy and/or lupin. In some embodiments, the vegetal base is or includes soy (e.g., soy plant matter).

In some embodiments, the vegetal base is or includes plant matter from nuts. Non-limiting examples of nuts contemplated for use herein include almonds, cashews, pecans, macadamias, hazelnuts, pistachio, walnuts, or combinations thereof.

In some embodiments, the vegetal base is or includes seeds. Non-limiting examples of seeds contemplated for use herein include hemp, pumpkin, quinoa, sesame, tiger nut, flax, chia, sunflower, coconut, or combinations thereof.

In some embodiments, the vegetal base is a milk of vegetal origin. For example, the milk is not a lacteal secretion obtained by milking any mammal, such as cows, sheep, goats, buffaloes, zebras, horses, donkeys, camels, and the like. In some embodiments, the vegetal base is or includes a plant-based milk. In some embodiments, the plant-based milk is or includes a milk of any of the plant matter described herein. For example, in some cases, the plant-based milk is a milk of a cereal, a legume, a nut, a seed, or a combination thereof. In some embodiments, the plant-based milk is a nut milk. In some embodiments, the plant-based milk is a cereal milk. In some embodiments, the plant-based milk is a legume milk. In some embodiments, the plant-based milk is a seed milk. In some embodiments, the plant-based milk is soy milk. In some embodiments, the vegetal base is a soy milk.

In some embodiments, the vegetal base is a suspension. In some embodiments, the vegetal base is an aqueous suspension containing water and plant matter as described herein. Methods of preparing a suspension of plant matter suitable for vegetal bases for fermentation as described herein are known in the art. Exemplary processes for preparing plant matter include mechanical and/or enzymatic disruption of the plant matter, optionally in combination with a solution or other form of hydration to produce an aqueous fraction which may be separate, e.g., decanted, centrifuged, and/or filtered, from the remaining plant matter, e.g., starchy, fibrous matter.

In some embodiments, the vegetal base is a butter alternative or beverage, such as a nut, seed, or pulse butter alternative or beverage. Methods for preparing butter alternatives for use as described herein are known in the art. Exemplary methods of preparing a butter alternative include wet or dry grinding roasted or unroasted e.g., nuts, seeds, pulses, to a paste. In some embodiments, the size of the particles of the paste are suitable for the preparation of beverages.

In some embodiments, the vegetal base does not include added sugars or proteins. For example, in some cases, the carbohydrate of the vegetal base is supplied only from the plant matter.

In some embodiments, the vegetal base includes proteins. In some embodiments, the proteins are supplied only from the plant matter. In some embodiments, the amount of protein is in the range of or of about 1 to 5% (% weight) of the total vegetal base. In some embodiments, the amount of protein is in the range of or of about 2 to 5% (% weight) of the total vegetal base. In some embodiments, the amount of protein is in the range of or of about 3 to 5% (% weight) of the total vegetal base. In some embodiments, the amount of protein is in the range of or of about 4 to 5% (% weight) of the total vegetal base. In some embodiments, the amount of protein is in the range of or of about 1 to 4% (% weight) of the total vegetal base. In some embodiments, the amount of protein is in the range of or of about 1 to 3% (% weight) of the total vegetal base. In some embodiments, the amount of protein is in the range of or of about 1 to 2% (% weight) of the total vegetal base.

In some embodiments, sugars may be added to the vegetal base. In some embodiments, the sugars are added in the range of or of about 0.5 to 5% (% weight) of the total vegetal base. In some embodiments, the sugars are added in the range of or of about 1 to 5% (% weight) of the total vegetal base. In some embodiments, the sugars are added in the range of or of about 2 to 5% (% weight) of the total vegetal base. In some embodiments, the sugars are added in the range of or of about 2 to 4% (% weight) of the total vegetal base. In some embodiments, the sugars are added in the range of or of about 2 to 3% (% weight) of the total vegetal base. In some embodiments, the sugars are added at or about 2% (% weight) of the total vegetal base. In some embodiments, the sugars are dextrose or sucrose. In some embodiments, the sugar is dextrose.

In some embodiments, the vegetal base has low, undetectable, or unquantifiable levels of acetaldehyde. In some embodiments, the vegetal base has low, undetectable, or unquantifiable levels of diacetyl. In some embodiments, the vegetal base has low, undetectable, or unquantifiable levels of acetaldehyde and diacetyl. Methods for detecting and/or quantifying levels of volatile organic compounds, such as acetaldehyde and diacetyl, include but are not limited to mass spectrometry, e.g., gas chromatograph-mass spectrometry (GC-MS), selected ion flow tube mass spectrometry (SIFT-MS), proton transfer reaction mass spectrometry (PTR-MS), ion-molecule reaction mass spectrometry (IMR-MS). In some embodiments, low levels of acetaldehyde or diacetyl are less than 0.5 ppm, e.g., less than 0.4, 0.3, 0.2, or 0.1 ppm. In some embodiments, low levels of acetaldehyde or diacetyl are between about 0.5 to about 0.1 ppm, about 0.4 to about 0.1 ppm, about 0.3 to about 0.1 ppm, or about 0.2 to about 0.1 ppm. In some embodiments, undetectable levels of acetaldehyde and diacetyl refer to amounts of the volatile organic compound that are below the lower limit for detection and/or quantification by the instrument used to detect the compound. In some embodiments, acetaldehyde and diacetyl are detected and quantified using GC-MS. In some embodiments, the lower limit for quantification is 0.125 ppm. In some embodiments, fermentation of the vegetal base according to the methods and compositions described herein results in an increase in an amount of acetaldehyde and/or diacetyl compared to the amount of acetaldehyde and/or diacetyl in the vegetal substrate prior to fermentation.

In some embodiments, the vegetal base is inoculated with bacteria or a bacterial composition, e.g., starter culture as described in Section I-B, to facilitate fermentation.

### B. Bacteria and Bacterial Compositions

The vegetal base, as described in Section I-A, may be inoculated with one or more bacterial strains, to facilitate fermentation. In some embodiments, the one or more bacteria are contained in a bacterial composition, such as a starter culture. In some embodiments, the one or more bacterial strains are contained in a starter culture. In some embodiments, the starter culture is a preparation of living bacteria able to assist in fermentation. In some embodiments, the vegetal base is inoculated with a starter culture. In some embodiments, the vegetal base is inoculated with a starter culture and a protective culture. In some embodiments, a protective culture is a culture able to reduce or prevent the growth of biological contaminants, such as yeast and mold. In some embodiments, the protective culture may include one or more bacterial strains described herein, e.g., L. rhamnosus strains described herein. The terms inoculating and adding may be used interchangeably to refer to contacting a vegetal base with one or more bacteria, for example as contained in a bacterial composition, such as a starter culture or a protective culture.

In some embodiments, the vegetal base is inoculated with one or more bacterial strains separately. For example, the bacterial strains are not mixed together prior to being added to the vegetal base. In some embodiments, the bacterial strains are mixed together prior to being added to the vegetal base. Regardless of how a bacterial strain is added to the vegetal base, the strain or mixtures of strains used for inoculation may be referred to as or included in a starter culture or protective culture. In some embodiments, the starter culture is a thermophilic starter culture, and the composition of the starter culture is suitable for thermophilic fermentation as described in Section I-C. In some embodiments, the starter culture includes a combination of bacteria suitable for mesophilic and/or thermophilic fermentation. In some embodiments, the starter culture includes mesophilic and thermophilic bacteria, and the composition of the starter culture is suitable for mesophilic and/or thermophilic fermentation, e.g., as described in Section I-C.

In some embodiments, thermophilic microorganisms, such as thermophilic bacteria, refer to microorganisms that function preferentially at temperatures above 37° C., e.g., 37 to 50° C. In some embodiments, mesophilic microorganisms, such as mesophilic bacteria, refer to microorganisms that function preferentially at temperatures at or below 37° C., e.g., 25 to 37° C.

In some embodiments, the starter culture is a pure culture, i.e., comprises or consists of a single bacterial strain. In some embodiments, the starter culture is a mixed culture, i.e. comprises or consists of at least one bacterial strain of the invention as described herein and at least one other bacterial strain. For example, at least 1 or more, and in particular 1, 2, 3, 4 or 5, other bacterial strains are included in the starter culture.

In some embodiments, the starter culture contains one or more lactic acid bacteria. In some embodiments, the starter culture contains at least two lactic acid bacteria. As it is normal in lactic acid bacterial fermentation processes to apply a mixed culture as a starter culture, the composition may in some embodiments include a multiplicity of strains either belonging to the same species or belonging to different species. For example, in some cases, the lactic acid bacteria in the starter culture is or includes a mixture of a Lactobacillus delbrueckii subsp bulgaricus strain, a Streptococcus thermophilus strain, and an L. rhamnosus strain.

In some embodiments, the starter culture includes at least bacteria from the genera Streptococcus and Lacticaseibacillus. In some embodiments, the starter culture includes or consists of bacteria from the genus Lactococcus, Lactobacillus, Streptococcus, Lacticaseibacillus, Leuconostoc, Pediococcus, Enterococcus, Bifidobacterium, Paralactobacillus, Acetilactobacillus, Agrilactobacillus, Amylolactobacillus, Apilactobacillus, Bombilactobacillus, Companilactobacillus, Dellaglioa, Fructilactobacillus, Furfurilactobacillus, Holzapfelia, Lacticaseibacillus, Lactiplantibacillus, Lapidilactobacillus, Latilactobacillus, Lentilactobacillus, Levilactobacillus, Ligilactobacillus, Limosilactobacillus, Liquorilactobacillus, Loigolactobacilus, Paucilactobacillus, Schleiferilactobacillus, Secundilactobacillus, or any combination thereof. In some embodiments, the starter culture includes or consists of bacteria from the genus Lactococcus, Lactobacillus, Streptococcus, Lacticaseibacillus, Leuconostoc, Pediococcus, Enterococcus or Bifidobacterium, or any combination thereof. In some embodiments, the starter culture includes or consists of bacteria from the genus Lactococcus, Lactobacillus, Streptococcus, Lacticaseibacillus, Leuconostoc, Pediococcus, or Bifidobacterium, or any combination thereof. In some embodiments, the starter culture includes or consists of bacteria from the genus Lactococcus, Lactobacillus, Streptococcus, Lacticaseibacillus, or any combination thereof. In some embodiments, the starter culture includes or consists of bacteria from the genus Lactobacillus, Streptococcus, Lacticaseibacillus, or any combination thereof. In some embodiments, the starter culture includes or consists of bacteria from the genus Streptococcus and Lacticaseibacillus.

In some embodiments, the starter culture includes at least a Streptococcus thermophilus strain and a Lacticaseibacillus rhamnosus strain. In some embodiments, the starter culture includes one or more of a Streptococcus thermophilus strain, a Lacticaseibacillus rhamnosus strain, a Lactobacillus acidophilus strain, a Bifidobacterium lactis strain, a Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, Lactococcus cremoris strain, or any combination of the foregoing. In some embodiments, the starter culture includes one or more of a Streptococcus thermophilus strain, a Lactobacillus acidophilus strain, a Lacticaseibacillus rhamnosus strain, a Bifidobacterium lactis strain, a Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, a Lactococcus lactis strain, a Lactococcus cremoris strain, or any combination of the foregoing. In some embodiments, the starter culture includes one or more of a Streptococcus thermophilus strain, a Lacticaseibacillus rhamnosus strain, a Lactobacillus delbrueckii subsp bulgaricus strain, or any combination of the foregoing. In some embodiments, the starter culture includes a Streptococcus thermophilus strain and a Lacticaseibacillus rhamnosus strain. In some embodiments, the starter culture includes more than one strain of a species.

In some embodiments, the starter culture includes at least one Lactococcus strain. For example, the starter culture may include one or more of a Lactococcus strain known in the art, such as a strain of Lactococcus cremoris subsp cremoris (previously referred to as Lactococcus lactis subsp. cremoris), Lactococcus lactis subsp hordniae, or Lactococcus lactis subsp lactis. In some embodiments, the starter culture includes a Lactococcus cremoris subsp cremoris and/or a Lactococcus lactis subsp lactis strain.

As described in the Examples, fermented plant-based compositions inoculated with starter cultures including Streptococcus thermophilus and Lacticaseibacillus rhamnosus strains had a diacetyl content greater than the content of acetaldehyde that correlated with increased cream flavor. Thus, in some embodiments, the starter culture is or includes strains of the species Streptococcus thermophilus and Lacticaseibacillus rhamnosus.

In some embodiments, the starter culture includes one or more Lacticaseibacillus rhamnosus strains. In some embodiments, the starter culture includes one or more Lacticaseibacillus rhamnosus strains of vegetal origin. For example, in some cases, Lacticaseibacillus rhamnosus strain is derived, selected, or isolated from plant material, e.g., fermented plant material. In some embodiments, the starter culture includes Lacticaseibacillus rhamnosus strain DGCC1179 that was deposited with the German Collection of Microorganisms and Cell Cultures (DSMZ) under accession no. DSM33650 or a mutant strain thereof. In some embodiments, the mutant strain is obtained by using the deposited strain as starting material. In some embodiments, the mutant strain is a strain having all of the identifying characteristics of the strain deposited at the DSMZ under number DSM33650. In some embodiments, the starter culture includes Lacticaseibacillus rhamnosus strain DGCC1460 that was deposited at the DSMZ under accession no. DSM22876 or a mutant strain thereof. In some embodiments, the mutant strain is obtained by using the deposited strain as starting material. In some embodiments, the mutant strain is a strain having all of the identifying characteristics of the strain deposited at the DSMZ under number DSM22876. In some embodiments, the starter culture includes Lacticaseibacillus rhamnosus strain DGCC13617 that was deposited at the DSMZ under accession no. DSM33850 or a mutant strain thereof. In some embodiments, the mutant strain is obtained by using the deposited strain as starting material. In some embodiments, the mutant strain is a strain having all of the identifying characteristics of the strain deposited at DSMZ under number DSM33850.

In some embodiments, the starter culture includes one or more Streptococcus thermophilus strains. In some embodiments, the starter culture includes Streptococcus thermophilus strain DGCC11042 that was deposited at the DSMZ under accession no. DSM33651 or a mutant strain thereof. In some embodiments, the mutant strain is obtained by using the deposited strain as starting material. In some embodiments, the mutant strain is a strain having all of the identifying characteristics of the strain deposited at DSMZ under number DSM33651.

In some embodiments, the starter culture includes one or more strains of Lacticaseibacillus rhamnosus and/or one or more strains of Streptococcus thermophilus. In some embodiments, the starter culture includes one or more strains of Lacticaseibacillus rhamnosus disclosed herein and/or one or more strains of Streptococcus thermophilus disclosed herein.

In some embodiments, the starter culture includes or consists of strain DSM33650 or a mutant thereof and strain DSM33651 or a mutant thereof. In some embodiments, the starter culture includes or consists of strain DSM22876 or a mutant thereof and strain DSM33651 or a mutant thereof. In some embodiments, the starter culture includes or consists of strain DSM33850 or a mutant thereof and strain DSM33651 or a mutant thereof. In some embodiments, the mutant strain is obtained by using the deposited strain as starting material. In some embodiments, the mutant is a strain having all of the identifying characteristics of the strain deposited at DSMZ.

As used herein, a mutant may be understood as a strain derived from a strain described herein by means of e.g., genetic engineering, radiation, UV light, and/or chemical treatment and/or methods that induce changes in the genome. The mutant may be a functionally equivalent mutant, e.g., a mutant that has substantially the same, or improved, properties (e.g., regarding diacetyl and acetaldehyde content regulation and/or production in fermented plant-based compositions; flavor; post-acidification; acidification speed; and/or phage robustness) as the parent strain. In some embodiments, the mutant decreases acetaldehyde content in a fermented plant-based compositions. In some embodiments, the mutant increases diacetyl content in a fermented plant-based compositions. In some embodiments, the mutant decreases acetaldehyde and/or increases diacetyl content in a fermented plant-based composition. In some embodiments, the mutant decreases acetaldehyde and increases diacetyl content in a fermented plant-based composition when used in combination with other strains or mutants. In some embodiments, the mutant either alone or in combination with another strain or mutant is useful for achieving levels of acetaldehyde and diacetyl, and relative quantities and ratios thereof, that correlate with a cream flavor, e.g., increased cream flavor, in a fermented plant-based composition. In some embodiments, the mutant may demonstrate phage resistance and/or reduced phage sensitivity that is the same or different from the phage resistance and/or reduced phage sensitivity of the parent strain. In some embodiments, the phage resistance and/or reduced phage sensitivity may result from differences at one or more CRISPR loci compared to the parent strain. In some embodiments, the difference may be the addition of one or more spacers in one or more CRISPR loci. In some embodiments, the phage resistance and/or reduced phage sensitivity may result from non-CRISPR-mediated phage resistance mechanisms that differ from those of the parent strain. Thus, in some embodiments, the CRISPR loci of the mutant strain are identical to the CRISPR loci of the parent strain but the mutant strain displays different phage resistances and/or reduced phage sensitivities compared to the parent strain. Methods for inducing or generating phage resistance or reduced sensitivity and detecting such features are known in the art. Non-limiting methods for inducing phage resistance or reduced phage sensitivity may be found, for example, in published international applications WO 2007/025097, WO 2007/136815, and WO 2008/108989, which are incorporated by reference in their entirety. Such mutants are a part of the present invention.

A mutant, e.g., mutant strain, as referred to herein may be a strain obtained by subjecting a strain described herein to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood as containing one mutagenization step followed by a screening/selection step). In some embodiments, no more than 20, or no more than 10, or no more than 5 treatments (or screening/selection steps) are carried out to produce a mutant. In some embodiments, less than 5%, or less than 1% or less than 0.1% of the nucleotides in the bacterial genome have been shifted with another nucleotide, deleted, substituted or added compared to the mother strain. In some embodiments, the mutant contains no more than 20, in particular no more than 10, in particular no more than 5, in particular no more than 4, in particular no more than 3, in particular no more than 2, and in particular no more than 1 nucleotide mutation of the bacterial genome as compared to the mother strain, wherein a mutation is a substitution, an insertion or a deletion of a nucleotide. In some embodiments, a mutant is obtained by subjecting a strain described herein to a selective pressure or a stressor.

In some embodiments, the starter culture, either as a pure or mixed culture as defined above, is in frozen, dried, freeze-dried, liquid or solid format, in the form of pellets or frozen pellets, or in a powder or dried powder. In some embodiments, the starter culture is in a frozen format or in the form of pellets or frozen pellets, in particular contained in one or more boxes or sachets. In some embodiments, the starter cultures as defined herein are in a powder form, such as a dried or freeze-dried powder, in particular contained in one or more boxes or sachets. In some embodiments, the liquid format is a bulk starter, such as a bacterial strain or mixture of bacterial strains previously propagated in a growth medium to obtain the required concentration for inoculation.

Commercial non-concentrated cultures of bacteria are referred to in industry as ‘mother cultures’, and are propagated at the production site before being added to a starting material, such as a vegetal base, for fermentation.

In some embodiments, the starter culture, either as a pure culture or mixed culture as defined above, and whatever the format (frozen, dried, freeze-dried, liquid or solid format, in the form of pellets or frozen pellets, or in a powder or dried powder) includes the bacterial strain(s) described herein in a concentration in the range of or of about 10⁵ to 10¹³ cfu (colony forming units) per gram of the starter culture. In some embodiments, the concentration of the bacterial strain(s) described herein within the starter culture of the invention is in the range of or of about 10⁵ to 10¹³ cfu per gram of the starter culture, and in particular at least 10⁶, at least 10⁷, at least 10⁸, at least 10⁹, at least 10¹⁰ or at least 10¹¹ CFU/g of the starter culture. In some embodiments, when in the form of frozen or dried concentrate, the concentration of bacterial strain(s) described herein—as a pure culture or as a mixed culture-within the starter culture is in the range of or of about 10⁶ to 10¹³ cfu/g of frozen concentrate or dried concentrate, and more preferably at least 10⁷, at least 10⁸, at least 10⁹, at least 10¹⁰, at least 10¹¹, at least 10¹², or at least 10¹³ cfu/g of frozen concentrate or dried concentrate. In some embodiments, the concentration of the bacterial strain(s) described herein within the starter culture of the invention is the range of or of about 10⁵ to 10⁹ CFU/g of the starter culture. In some embodiments, the concentration of the bacterial strain(s) described herein within the starter culture of the invention is the range of or of about 10⁶ to 10⁹ CFU/g of the starter culture. In some embodiments, the concentration of the bacterial strain(s) described herein within the starter culture of the invention is the range of or of about 10⁶ to 10⁸ CFU/g of the starter culture. In some embodiments, the concentration of the bacterial strain(s) described herein within the starter culture of the invention is the range of or of about 10⁶ to 10⁷ CFU/g of the starter culture. In some embodiments, the concentration of the bacterial strain(s) described herein within the starter culture of the invention is, is at least, or is about 10⁶ CFU/g of the starter culture. In some embodiments, the concentration of the bacterial strain(s) described herein within the starter culture of the invention is, is at least, or is about 10⁷ CFU/g of the starter culture.

Starter cultures may be prepared by techniques well known in the art such as those disclosed in U.S. Pat. No. 4,621,058. By way of example, starter cultures may be prepared by the introduction of an inoculum, for example a bacterium, to a growth medium to produce an inoculated medium and ripening the inoculated medium to produce a starter culture. Dried starter cultures may be prepared by techniques well known in the art, such as those discussed in U.S. Pat. Nos. 4,423,079 and 4,140,800. In some embodiments, the starter culture is in the form of concentrated frozen pellets.

In some embodiments, the addition of the starter culture to the vegetal base is direct, e.g., as a direct vat inoculate, direct vat starter, direct vat set culture.

In some embodiments, the vegetal base may be inoculated with the starter culture prior to initiating fermentation. In some embodiments, the vegetal base is inoculated with the starter culture following, e.g., at a time point after, fermentation is initiated. In some embodiments, the vegetal base is inoculated with the starter culture during fermentation. For example, in some cases where strains of the starter culture are added separately, e.g., not in a mixture, the strains may be added at different times before or during fermentation.

Further microorganisms and/or additives may be added to the vegetal base before, during or after fermentation of the vegetal base. For example, boosters, such as yeast extracts and/or amino acid containing compositions may be added to the vegetal base in addition to a starter culture, to support or promote microorganism function and facilitate successful production of the fermented plant-based compositions. Microorganisms that may be added to the vegetal base include those that will contribute in an advantageous manner to the properties of the fermented plant-based compositions, e.g., fermented plant-based food products. For example, added microorganisms, yeast extracts, or amino acid compositions may improve or support diacetyl production, acetaldehyde conversion and breakdown, the viscosity, gel stiffness, mouth coating, flavor, post acidification, and/or acidification speed in the fermented plant-based compositions. Optionally, other ingredients may be added to the vegetal base, such as colors, stabilizers, e.g., pectin, starch, modified starch, CMC, etc.; or polyunsaturated fatty acids, e.g. omega-3 fatty acids. Such ingredients may be added at any point during the production process, e.g. before or after fermentation.

Components such as cryoprotectants and/or conventional additives including nutrients such as yeast extracts, sugars and vitamins, e.g., vitamin A, C, D, K or vitamins of the vitamin B family may also be added to the vegetal base either with or separate from the starter culture or other additives. Suitable cryoprotectants that may be added include components that improve the cold tolerance of the microorganisms, such as mannitol, sorbitol, sodium tripolyphosphate, xylitol, glycerol, raffinose, maltodextrin, erythritol, threitol, trehalose, glucose and fructose. Other additives may include carbohydrates, flavors, minerals, enzymes (e.g., rennet, lactase and/or phospholipase).

In some embodiments, additives, microorganisms, or other components as described in the preceding paragraphs are added simultaneously or nearly simultaneously with the starter culture to the vegetal base, e.g., at the same time as inoculation of the vegetal base with the starter culture. In some embodiments, additives, microorganisms, or other components as described in the preceding paragraphs are added to the vegetal base before, e.g., temporally prior to, inoculation of the vegetal base with the starter culture. In some embodiments, additives, microorganisms, or other components as described in the preceding paragraphs are added after, e.g., temporally following, inoculation of the vegetal base with the starter culture. In some embodiments, additives, microorganisms, or other components as described in the preceding paragraphs may be added to the vegetal base before, during, or after fermentation, for example independent of the inoculation with the starter culture.

In some embodiments, the vegetal base is inoculated with the starter culture, and optionally additives, microorganisms, or other components as described in the preceding paragraphs above, by any suitable method. For example, the vegetal base may be inoculated by direct inoculation into a fermentation vessel.

### C. Fermentation

The methods for producing a fermented plant-based composition having cream flavor provided herein include fermentation, e.g., thermophilic fermentation, of a vegetal base, for example as described in Section I-A, inoculated with a starter culture, and optionally other additives, microorganisms, and components, as described in Section I-B.

Fermentation according to the methods provided herein facilitates the conversion of carbohydrates into alcohols or acids through the action of a microorganism, e.g., bacteria present, for example, in a starter culture. In some embodiments, fermentation according to the methods provided herein includes conversion of lactose to lactic acid.

Fermentation processes may be selected to optimize parameters such as temperature, oxygen, process time to achieve a fermented plant-based compositions having cream flavor as described herein. In some embodiments, the fermentation is mesophilic fermentation. In some embodiments, the fermentation is thermophilic fermentation.

In some embodiments, the inoculated vegetal base is fermented at a temperature at or above about 25° C., 30° C., 35° C., 40° C., or 50° C. In some embodiments, the inoculated vegetal base is fermented at a temperature of between or between about 20 to 50° C., 30 to 50° C., or 40 to 50° C. In some embodiments, the inoculated vegetal base is fermented at a temperature of above or of about 30° C., 33° C., 35° C., 36° C., 37° C., 38° C., 39° C., 40° C., 41° C., 42° C., 43° C., 44° C., 45° C., or 50° C. In some embodiments, the inoculated vegetal base is fermented at a temperature of between or between about 35 to 47° C., 35 to 46° C., 35 to 45° C., 35 to 44° C., 35 to 43° C., 36 to 43° C., 37 to 43° C., 38 to 43° C., 39 to 43° C., 40 to 43° C., 41 to 43° C., or 42 to 43° C. In some embodiments, the inoculated vegetal base is fermented at a temperature of or of about 36° C., 37° C., 38° C., 39° C., 40° C., 41° C., 42° C., 43° C., or 44° C. In some embodiments, the inoculated vegetal base is fermented at a temperature of or of about 37° C., e.g., 37±1° C. In some embodiments, the inoculated vegetal base is fermented at a temperature of or of about 40° C., e.g., 40±1° C. In some embodiments, the inoculated vegetal base is fermented at a temperature of or of about 43° C., e.g., 43±1° C. In some embodiments, more than one temperature or range of temperatures may be used for fermentation.

In some embodiments, fermentation of the inoculated vegetal base is carried out for at least 4 hours, e.g., 5, 6, 7, 8, 9, 10, 15, 20, or 24 hours. In some embodiments, fermentation of the inoculated vegetal base is carried out for or for about 4, 5, 6, 7, 8, 9, 10, 11, 12, 13, 14, 15, 16, 17, 18, 19, 20, 21, 22, 23, or 24 hours. In some embodiments, fermentation of the inoculated vegetal base is carried out for at most 24 hours. In some embodiments, fermentation of the inoculated vegetal base is carried out for between or between about 4 to 24, 4 to 23, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, or 4 to 5 hours. In some embodiments, fermentation of the inoculated vegetal base is carried out for or for about 10 hours. In some embodiments, fermentation of the inoculated vegetal base is carried out for or for about 9 hours. In some embodiments, fermentation of the inoculated vegetal base is carried out for or for about 8 hours. In some embodiments, fermentation of the inoculated vegetal base is carried out for or for about 7 hours. In some embodiments, fermentation of the inoculated vegetal base is carried out for or for about 6 hours. In some embodiments, fermentation of the inoculated vegetal base is carried out for or for about 5 hours. In some embodiments, fermentation of the inoculated vegetal base is carried out for or for about 4 hours.

In some embodiments, fermentation of the inoculated vegetal base is carried out for an amount of time needed for the vegetal base to reach a target pH. In some embodiments, the target pH is a suitable pH for producing the fermented plant-based composition that are food products, e.g., as described in Section I-F below. In some embodiments, the target pH is in a range of or of about 3.4 to 5. In some embodiments, the target pH is in a range of or of about 3.8 to 5. In some embodiments, the target pH is in a range of or of about 4 to 5. In some embodiments, the target pH is or is about 4.1, 4.2, 4.3, 4.4, 4.5, 4.6, or 4.7. In some embodiments, the target pH is in a range of or of about 4.2 to 4.7. In some embodiments, the target pH is or is about 4.6.

At the end of fermentation, in some embodiments, the fermented plant-based composition has a pH in the range of or of about 3.4 to 5. In some embodiments, the fermented plant-based composition has a pH in the range of or of about 3.8 to 5 at the end of fermentation. In some embodiments, the fermented plant-based composition has a pH in the range of or of about 4 to 5 at the end of fermentation. In some embodiments, the fermented plant-based composition has a pH of or of about 4.1, 4.2, 4.3, 4.4, 4.5, 4.6, or 4.7 at the end of fermentation. In some embodiments, the fermented plant-based composition has a pH in the range of or of about 4.2 to 4.7 at the end of fermentation. In some embodiments, the fermented plant-based composition has a pH of or of about 4.6 at the end of fermentation.

In some embodiments, the pH of the inoculated vegetal base is measured, e.g., monitored, during fermentation. In some embodiments, the pH of the inoculated vegetal base is measured, e.g., monitored, at specific time points during fermentation. For example, a pH measurement may be taken every 5, 10, 15, 20, 25, 30, 35, 40, 45, 50, 55, or 60 minutes, or every hour, 2 hours, 3 hours, 4 hours, or 5 hours. In some embodiments, the pH of the inoculated vegetal base is measured, e.g., monitored, continuously during fermentation.

In some embodiments, the fermented plant-based composition, produced by fermentation of a vegetal base with a starter culture as described herein, contains levels of acetaldehyde and/or diacetyl that are increased compared to the levels of acetaldehyde and/or diacetyl in the vegetal base prior to fermentation.

### D. Storage

In some aspects, the fermented plant-based composition produced according to the methods provided herein is stored. For example, in some embodiments, following fermentation of the inoculated vegetal base as described herein (see, Sections I-A, I-B, and I-C), the fermented plant-based composition is stored to produce a stored fermented plant-based composition.

In some embodiments, the fermented plant-based composition is stored in one or more containers. Suitable containers for storage included, but are not limited to, containers capable of securing and protecting the fermented plant-based composition, e.g., protecting the fermented plant-based composition from contamination. In some embodiments, the fermented plant-based composition is packaged in a sealed container. In some embodiments, the fermented plant-based composition is packaged in a container after fermentation has occurred. In some embodiments, the fermentation occurs in a container that can be sealed. For example, in some cases, a vegetal base, e.g., inoculated vegetal base, may be fermented in a container such that at the end of fermentation the container contains a plant-based fermented composition and the container is then sealed. In some embodiments, the vegetal base is inoculated prior to placement in the container for fermentation. In some embodiments, the vegetal base is inoculated after to placement in the container for fermentation.

In some embodiments, the one or more containers are useful for storing the fermented plant-based composition at a cool temperature, e.g., a cooler temperature than used for fermentation, such as at or at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or 26° C. In some embodiments, the fermented plant-based composition is stored at a temperature in the range of or of about 1 to 26° C., 1 to 24° C., 1 to 22° C., 1 to 20° C., 1 to 18° C., 1 to 16° C., 1 to 14° C., 1 to 12° C., 1 to 10° C., 1 to 8° C., 1 to 6° C., 1 to 4° C., or 1 to 2° C. In some embodiments, the fermented plant-based composition is stored at a temperature in the range of or of about 1 to 14° C., 1 to 12° C., 1 to 10° C., 1 to 8° C., 1 to 6° C., or 1 to 4° C. In some embodiments, the fermented plant-based composition is stored at a temperature in the range of or of about 4 to 8° C. In some embodiments, the fermented plant-based composition is stored at or at about a temperature of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or 26° C. In some embodiments, the fermented plant-based composition is stored at or at about a temperature of 1° C., 2° C., 3° C., 4° C., 5° C., 6° C., 7° C., 8° C., 9° C., 10° C., 11° C., 12° C., 13° C. or 14° C. In some embodiments, the fermented plant-based composition is stored at or at about a temperature of 2° C., 3° C., 4° C., 5° C., 6° C., 7° C., or 8° C. In some embodiments, the fermented plant-based composition is stored at or at about a temperature of 6° C., e.g., 6±1° C.

In some embodiments, stored fermented plant-based composition maintains a target pH achieved during fermentation. In some embodiments, the target pH is a suitable pH for producing the fermented plant-based composition that is a food product, e.g., as described in Section I-F below. In some embodiments, the target pH is in a range of or of about 3.8 to 5. In some embodiments, the target pH is in a range of or of about to 5. In some embodiments, the target pH is or is about 4.1, 4.2, 4.3, 4.4, 4.5, 4.6, or 4.7. In some embodiments, the target pH is in a range of or of about 4.2 to 4.7. In some embodiments, the target pH is or is about 4.6.

In some embodiments, the stored fermented plant-based composition has a pH in the range of or of about 3.4 to 4.5. In some embodiments, the stored fermented plant-based composition has a pH in the range of or of about 3.8 to 4.2. In some embodiments, the stored fermented plant-based composition has a pH of or of about 3.8, 3.9, 4.0, 4.1, 4.2, or 4.3. In some embodiments, the stored fermented plant-based composition has a pH in the range of or of about 3.9 to 4.1. In some embodiments, the stored fermented plant-based composition has a pH of or of about 4.0.

In some embodiments, the pH of the stored fermented plant-based composition is measured, e.g., monitored, during storage. In some embodiments, the pH of the stored fermented plant-based composition is measured, e.g., monitored, at specific time points during storage. For example, a pH measurement may be taken every or every other day, every third, every fourth, every fifth, every sixth, every seventh, every eighth, every ninth, every tenth, every eleventh, or every twelfth, every thirteenth, or every fourteenth day in storage for the duration of storage. In some embodiments, the pH of the stored fermented plant-based composition is measured, e.g., monitored at about every 5 hours, 10 hours, 15 hours, 20 hours, 24 hours, 36 hours, 48 hours, 60 hours, or every 72 for the duration of time in storage. In some embodiments, the pH of the stored fermented plant-based composition is measured, e.g., monitored, continuously during storage. In some embodiments, the pH of the stored fermented plant-based composition is measured at a temperature of or of about 20° C. For example, the stored fermented plant-based composition may be removed from storage for a period of time to increase the temperature of the stored fermented plant-based composition prior to measuring the pH.

In some embodiments, the fermented plant-based composition, e.g., stored fermented plant-based composition, is stored for at least or at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days. In some embodiments, the fermented plant-based composition, e.g., stored fermented plant-based composition, is stored for at least or at least about 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days. In some embodiments, the fermented plant-based composition, e.g., stored fermented plant-based composition, is stored for at least or at least about 7, 8, 9, 10, 11, 12, 13, 14 15, 16, 17, 18, 19, 20, 21, 22, 23, 24, 25, 26, 27, 28, 29, 30, 40, 50, 60, 70, 80, or 90 days. In some embodiments, the fermented plant-based composition, e.g., stored fermented plant-based composition, is stored for at least or at least about 7, 8, 9, 10, 11, 12, 13, 14 15, 16, 17, 18, 19, 20, 21, 22, 23, 24, 25, 26, 27, 28, 29, 30, 40, 50, or 60 days. In some embodiments, the fermented plant-based composition, e.g., stored fermented plant-based composition, is stored for at least or at least about 7, 8, 9, 10, 11, 12, 13, 14 15, 16, 17, 18, 19, 20, 21, 22, 23, 24, 25, 26, 27, 28, 29, or 30 days. In some embodiments, the fermented plant-based composition, e.g., stored fermented plant-based composition, is stored for at least or at least about 4, 3, 2, or 1 month. In some embodiments, the fermented plant-based composition, e.g., stored fermented plant-based composition, is stored for at least or at least about 120 days. In some embodiments, the fermented plant-based composition, e.g., stored fermented plant-based composition, is stored for at least or at least about 90 days. In some embodiments, the fermented plant-based composition, e.g., stored fermented plant-based composition, is stored for at least or at least about 60 days. In some embodiments, the fermented plant-based composition, e.g., stored fermented plant-based composition, is stored for at least or at least about 30 days. In some embodiments, the fermented plant-based composition, e.g., stored fermented plant-based composition, is stored for at least or at least about 28 days. In some embodiments, the fermented plant-based composition, e.g., stored fermented plant-based composition, is stored for at least or at least about 21 days. In some embodiments, the fermented plant-based composition, e.g., stored fermented plant-based composition, is stored for at least or at least about 14 days. In some embodiments, the fermented plant-based composition, e.g., stored fermented plant-based composition, is stored for at least or at least about 7 days.

In some embodiments, the fermented plant-based composition, e.g., stored fermented plant-based composition, is stored for at most or at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days. In some embodiments, the fermented plant-based composition, e.g., stored fermented plant-based composition, is stored for at most or at most about 7, 8, 9, 10, 11, 12, 13, 14 15, 16, 17, 18, 19, 20, 21, 22, 23, 24, 25, 26, 27, 29, 30, 40, 50, 60, 70, 80, 90, 100, 110, or 120 days. In some embodiments, the fermented plant-based composition, e.g., stored fermented plant-based composition, is stored for at most or at most about 7, 8, 9, 10, 11, 12, 13, 14 15, 16, 17, 18, 19, 20, 21, 22, 23, 24, 25, 26, 27, 29, 30, 40, 50, 60, 70, 80, or 90 days. In some embodiments, the fermented plant-based composition, e.g., stored fermented plant-based composition, is stored for at most or at most about 7, 8, 9, 10, 11, 12, 13, 14 15, 16, 17, 18, 19, 20, 21, 22, 23, 24, 25, 26, 27, 28, 29, 30, 40, 50, or 60 days. In some embodiments, the fermented plant-based composition, e.g., stored fermented plant-based composition, is stored for at most or at most about 7, 8, 9, 10, 11, 12, 13, 14 15, 16, 17, 18, 19, 20, 21, 22, 23, 24, 25, 26, 27, 28, 29, or 30 days. In some embodiments, the fermented plant-based composition, e.g., stored fermented plant-based composition, is stored for at most or at most about 4, 3, 2, or 1 month. In some embodiments, the fermented plant-based composition, e.g., stored fermented plant-based composition, is stored for at most or at most about 90 days. In some embodiments, the fermented plant-based composition, e.g., stored fermented plant-based composition, is stored for at most or at most about 60 days. In some embodiments, the fermented plant-based composition, e.g., stored fermented plant-based composition, is stored for at most or at most about 30 days. In some embodiments, the fermented plant-based composition, e.g., stored fermented plant-based composition, is stored for at most or at most about 28 days. In some embodiments, the fermented plant-based composition, e.g., stored fermented plant-based composition, is stored for at most or at most about 21 days. In some embodiments, the fermented plant-based composition, e.g., stored fermented plant-based composition, is stored for at most or at most about 14 days. In some embodiments, the fermented plant-based composition, e.g., stored fermented plant-based composition, is stored for at most or at most about 7 days.

In some embodiments, the fermented plant-based composition, e.g., stored fermented plant-based composition, is stored for a duration of or of about 1 to 120, 1 to 90, 1 to 60, 1 to 30, 1 to 25, 1 to 20, 1 to 15, 1 to 10, or 1 to 5 days. In some embodiments, the fermented plant-based composition, e.g., stored fermented plant-based composition, is stored for a duration of or of about 7 to 120, 7 to 90, 7 to 60, or 7 to 30 days. In some embodiments, the fermented plant-based composition, e.g., stored fermented plant-based composition, is stored for a duration of or of about 7 to 28, 7 to 21, or 7 to 14 days. In some embodiments, the fermented plant-based composition, e.g., stored fermented plant-based composition, is stored for a duration of or of about 7 to 120 days. In some embodiments, the fermented plant-based composition, e.g., stored fermented plant-based composition, is stored for a duration of or of about 7 to 90 days. In some embodiments, the fermented plant-based composition, e.g., stored fermented plant-based composition, is stored for a duration of or of about 7 to 60 days. In some embodiments, the fermented plant-based composition, e.g., stored fermented plant-based composition, is stored for a duration of or of about 7 to 30 days. In some embodiments, the fermented plant-based composition, e.g., stored fermented plant-based composition, is stored for a duration of or of about 7 to 21 or 7 to 14 days. In some embodiments, the fermented plant-based composition, e.g., stored fermented plant-based composition, is stored for a duration of or of about 120 days. In some embodiments, the fermented plant-based composition, e.g., stored fermented plant-based composition, is stored for a duration of or of about 90 days. In some embodiments, the fermented plant-based composition, e.g., stored fermented plant-based composition, is stored for a duration of or of about 60 days. In some embodiments, the fermented plant-based composition, e.g., stored fermented plant-based composition, is stored for a duration of or of about 30 days. In some embodiments, the fermented plant-based composition, e.g., stored fermented plant-based composition, is stored for a duration of or of about 28 days. In some embodiments, the fermented plant-based composition, e.g., stored fermented plant-based composition, is stored for a duration of or of about 21 days. In some embodiments, the fermented plant-based composition, e.g., stored fermented plant-based composition, is stored for a duration of or of about 14 days. In some embodiments, the fermented plant-based composition, e.g., stored fermented plant-based composition, is stored for a duration of or of about 7 days.

In some embodiments, the fermented plant-based composition, e.g., stored fermented plant-based composition, is stored for an amount of time or at least an amount of time suitable for the levels of acetaldehyde and diacetyl in the fermented plant-based composition, e.g., stored fermented plant-based composition, to reach a relative quantity or ratio correlated with a cream flavor, e.g., an increased cream flavor. In some embodiments, the fermented plant-based composition, e.g., stored fermented plant-based composition, is stored until a relative quantity or ratio of acetaldehyde to diacetyl as described in Section I-E is reached. In some embodiments, the fermented plant-based composition, e.g., stored fermented plant-based composition, is stored until a cream flavor, e.g., an increased cream flavor, as described in Section I-E is reached.

### E. Assessing Acetaldehyde, Diacetyl, and Cream Flavor

In some aspects, the fermented plant-based compositions produced according to the methods provided herein have cream flavor, such as an increased cream flavor. In some embodiments, the cream flavor is a cream aroma and/or cream odor. Without being bound by theory, the cream flavor may be correlated with the relative quantities of diacetyl and acetaldehyde present in the fermented plant-based composition. Thus, in some embodiments, the methods provided herein include decreasing acetaldehyde and increasing diacetyl content in the fermented plant-based composition to levels correlated with the perception of cream flavor, e.g., increased levels of cream aroma and/or cream odor, in the fermented plant-based composition. In some embodiments, the levels of acetaldehyde and diacetyl achieved according to the methods provided herein produce an increased cream flavor, e.g., cream aroma and/or cream odor, compared to other methods of producing a fermented plant-based composition that do not result in a relative content of acetaldehyde and diacetyl as described herein. In some embodiments, the increased cream flavor is an increased cream aroma and/or increased cream odor, e.g., as compared to fermented plant-based compositions produced via alternative methods.

Methods for detecting and optionally quantifying levels of volatile organic compounds, such as acetaldehyde and diacetyl, include but are not limited to, mass spectrometry, e.g., gas chromatograph-mass spectrometry (GC-MS), selected ion flow tube mass spectrometry (SIFT-MS), proton transfer reaction mass spectrometry (PTR-MS), ion-molecule reaction mass spectrometry (IMR-MS). In some embodiments, acetaldehyde and diacetyl in the fermented plant-based composition are detected and quantified using GC-MS.

Methods for determining organoleptic profiles, including cream flavor, e.g., cream aroma, cream odor, are known in the art, and include, for example, the use of trained panels of sensory assessors to perform quantitative descriptive analysis (QDA). In some embodiments, the trained sensory assessors evaluate descriptors using a linear scale, e.g., 7-point linear scale. In some embodiments, the descriptors evaluated include cream aroma and cream odor. In some embodiments, the QDA is performed blind, e.g., the assessors do not know the identity or methods of producing the assessed fermented plant-based compositions.

In some embodiments, the methods of producing a fermented plant-based composition having cream flavor include decreasing acetaldehyde content in the fermented plant-based composition. In some embodiments, the methods of producing a fermented plant-based composition having cream flavor include decreasing acetaldehyde content and increasing diacetyl content in the fermented plant-based composition. In some embodiments, the decrease in acetaldehyde results in a ratio of acetaldehyde to diacetyl that correlates with a cream flavor, e.g., increased cream flavor. In some embodiments, the decrease in acetaldehyde and the increase in diacetyl results in a ratio of acetaldehyde to diacetyl that correlates with a cream flavor, e.g., increased cream flavor.

The acetaldehyde content may be considered to decrease with respect to an initial acetaldehyde content. In some embodiments, the initial acetaldehyde content is the acetaldehyde content of the fermented plant-based composition. In some embodiments, the initial acetaldehyde content is the acetaldehyde content of the fermented plant-based composition at the end of the fermentation process. In some embodiments, the initial acetaldehyde content is the acetaldehyde content of the fermented plant-based composition at the initiation of storage, e.g., the first day in storage. Similarly, the diacetyl content may be considered to increase with respect to an initial diacetyl content. In some embodiments, the initial diacetyl content is the diacetyl content of the fermented plant-based composition at the end of the fermentation process. In some embodiments, the initial diacetyl content is the diacetyl content of the fermented plant-based composition at the initiation of storage, e.g., the first day in storage.

In some embodiments, the method of producing a fermented plant-based composition having cream flavor includes decreasing a level of acetaldehyde in the fermented plant-based composition compared to the level of acetaldehyde, e.g., initial acetaldehyde content, present in the fermented plant-based composition produced by fermentation of the inoculated vegetal base. In some embodiments, the method of producing a fermented plant-based composition having cream flavor includes increasing a level of diacetyl in the fermented plant-based composition compared to the level of diacetyl, e.g., initial diacetyl content, present in the fermented plant-based composition produced by fermentation of the inoculated vegetal base. In some embodiments, the method of producing a fermented plant-based composition having cream flavor includes decreasing a level of acetaldehyde in the fermented plant-based composition compared to the level of acetaldehyde, e.g., initial acetaldehyde content, present in the fermented plant-based composition produced by fermentation of the inoculated vegetal base and increasing a level of diacetyl in the fermented plant-based composition compared to the level of diacetyl, e.g., initial diacetyl content, present in the fermented plant-based composition produced by fermentation of the inoculated vegetal base. In some embodiments, the method of producing a fermented plant-based composition having cream flavor includes decreasing a level of acetaldehyde in the fermented plant-based composition compared to the level of acetaldehyde, e.g., initial acetaldehyde content, present in the fermented plant-based composition produced by fermentation of the inoculated vegetal base and/or increasing a level of diacetyl in the fermented plant-based composition compared to the level of diacetyl, e.g., initial diacetyl content, present in the fermented plant-based composition produced by fermentation of the inoculated vegetal base to produce a ratio of acetaldehyde to diacetyl correlated with a cream flavor, e.g., increased cream flavor, as described herein.

In some embodiments, the acetaldehyde content is decreased by a factor of at least 1.5, e.g., 2, 3, 4, 5, 6, 7, 8, 9, 10, 11, 12, 13, 14, 15, 20, 30, 40, or 50. In some embodiments, the acetaldehyde content is decreased by a factor of at least 2. In some embodiments, the acetaldehyde content is decreased by a factor in the range of or of about 1.5 to 15, 1.5 to 10, 1.5 to 9, 1.5 to 8, 1.5 to 7, 1.5 to 6, 1.5 to 5, 1.5 to 4, 1.5 to 3, or 1.5 to 2. In some embodiments, the acetaldehyde content is decreased by a factor in the range of or of about 1.5 to 10 ppm, 1.5 to 9, 1.5 to 8, 1.5 to 7, or 1.5 to 6. In some embodiments, the acetaldehyde content is decreased by a factor in the range of or of about 2 to 10. In some embodiments, the acetaldehyde content is decreased by a factor of 1.5, 2, 3, 4, 5, 6, 7, 8, 9, 10, 11, 12, 13, 14, 15, or 20. In some embodiments, the acetaldehyde content is decreased by a factor of or of about 1.5, 2, 3, 4, 5, 6, 7, 8, 9, 10, 11, 12, 13, 14, or 15. In some embodiments, the acetaldehyde content is decreased by a factor of or of about 1.5, 2, 3, 4, or 5. In some embodiments, the acetaldehyde content is decreased by a factor of or of about 1.5. In some embodiments, the acetaldehyde content is decreased by a factor of or of about 2.

In some embodiments, the method of producing a fermented plant-based composition having cream flavor includes decreasing the acetaldehyde content by a factor described herein.

In some embodiments, the acetaldehyde content is decreased to or to less than 1 ppm, 0.5 ppm, 0.4 ppm, 0.3 ppm, 0.2 ppm, 0.1 ppm, 0.09 ppm, 0.08 ppm, 0.07 ppm, 0.06 ppm, 0.05 ppm, 0.03 ppm, 0.02 ppm, 0.01 ppm or less. In some embodiments, the acetaldehyde content is decreased to a range of or of about 0.8 ppm to about 0 ppm, e.g., below the lower limit of quantification and/or detection, 0.7 ppm to about 0 ppm, e.g., below the lower limit of quantification and/or detection, 0.6 ppm to about 0 ppm, e.g., below the lower limit of quantification and/or detection, 0.5 ppm to about 0 ppm, e.g., below the lower limit of quantification and/or detection, 0.4 ppm to about 0 ppm, e.g., below the lower limit of quantification and/or detection, 0.3 ppm to about 0 ppm, e.g., below the lower limit of quantification and/or detection, 0.2 ppm to about 0 ppm, e.g., below the lower limit of quantification and/or detection, or 0.1 ppm to about 0 ppm, e.g., below the lower limit of quantification and/or detection. In some embodiments, the acetaldehyde content is decrease to about or below the lower limit of quantification and/or detection. In some embodiments, the acetaldehyde content is decreased to a range of or of about 0.6 ppm to about 0 ppm, e.g., below the lower limit of quantification and/or detection. In some embodiments, the acetaldehyde content is decreased to a range of or of about 0.2 ppm to about 0 ppm, e.g., below the lower limit of quantification and/or detection. In some embodiments, the acetaldehyde content in the fermented plant-based compositions is negligible. In some embodiments, the lower limit of detection and/or quantification is specific to the instrument used to detect the compound, e.g., acetaldehyde. In some embodiments, acetaldehyde is detected and quantified using GC-MS. In some embodiments, the lower limit for quantification is 0.125 ppm.

In some embodiments, the methods of producing a fermented plant-based composition having cream flavor produce an acetaldehyde content in the fermented plant-based composition that is or is less than 1 ppm, 0.9 ppm, 0.8 ppm, 0.7 ppm, 0.6 ppm, 0.5 ppm, 0.4 ppm, 0.3 ppm, 0.2 ppm, 0.1 ppm, 0.09 ppm, 0.08 ppm, 0.07 ppm, 0.06 ppm, 0.05 ppm, 0.03 ppm, 0.02 ppm, 0.01 ppm. In some embodiments, the acetaldehyde content produced is in a range of or of about 0.8 ppm to about 0 ppm, e.g., below the lower limit of quantification and/or detection, 0.7 ppm to about 0 ppm, e.g., below the lower limit of quantification and/or detection, 0.6 ppm to about 0 ppm, e.g., below the lower limit of quantification and/or detection, 0.5 ppm to about 0 ppm, e.g., below the lower limit of quantification and/or detection, 0.4 ppm to about 0 ppm, e.g., below the lower limit of quantification and/or detection, 0.3 ppm to about 0 ppm, e.g., below the lower limit of quantification and/or detection, 0.2 ppm to about 0 ppm, e.g., below the lower limit of quantification and/or detection, or 0.1 ppm to about 0 ppm, e.g., below the lower limit of quantification and/or detection. In some embodiments, the acetaldehyde content produced is, is about, or below the lower limit of quantification and/or detection. In some embodiments, the acetaldehyde content is decreased to a range of or of about 0.6 ppm to about 0 ppm, e.g., below the lower limit of quantification and/or detection. In some embodiments, the acetaldehyde content produced is in a range of or of about 0.2 ppm to about 0 ppm, e.g., below the lower limit of quantification and/or detection. In some embodiments, the acetaldehyde content produced in the fermented plant-based compositions is negligible. In some embodiments, the lower limit of detection and/or quantification is specific to the instrument used to detect the compound, e.g., acetaldehyde. In some embodiments, acetaldehyde is detected and quantified using GC-MS. In some embodiments, the lower limit for quantification is 0.125 ppm.

In some embodiments, the method of producing a fermented plant-based composition having cream flavor includes decreasing the acetaldehyde content to an amount described herein.

In some embodiments, the diacetyl content is increased by a factor of at least 1.5, e.g., 2, 5, 6, 7, 8, 9, 10, 11, 12, 13, 14, or 15. In some embodiments, the diacetyl content is increased by a factor of at least 2. In some embodiments, the diacetyl content is increased by a factor in the range of or of about 1.5 to 20, 1.5 to 15, 1.5 to 10, 1.5 to 9, 1.5 to 8, 1.5 to 7, 1.5 to 6, 1.5 to 5, 1.5 to 4, 1.5 to 3, or 1.5 to 2. In some embodiments, the diacetyl content is increased by a factor in the range of or of about 1.5 to 15, 1.5 to 10 ppm, 1.5 to 9, 1.5 to 8, 1.5 to 7, or 1.5 to 6. In some embodiments, the diacetyl content is increased by a factor in the range of or of about 1.5 to 10. In some embodiments, the diacetyl content is increased by a factor of 1.5, 2, 3, 4, 5, 6, 7, 8, 9, 10, 11, 12, 13, 14, 15, or 20. In some embodiments, the diacetyl content is increased by a factor of or of about 1.5, 2, 3, 4, 5, 6, 7, 8, 9, 10, 11, 12, 13, 14, or 15. In some embodiments, the diacetyl content is increased by a factor of or of about 1.5, 2, 3, 4, or 5. In some embodiments, the diacetyl content is increased by a factor of or of about 1.5. In some embodiments, the diacetyl content is increased by a factor of or of about 2. In some embodiments, the diacetyl content is increased by a factor of or of about 3. In some embodiments, the diacetyl content is increased by a factor of or of about 4. In some embodiments, the diacetyl content is increased by a factor of or of about 5. In some embodiments, the diacetyl content is increased by a factor of or of about 6. In some embodiments, the diacetyl content is increased by a factor of or of about 7. In some embodiments, the diacetyl content is increased by a factor of or of about 8. In some embodiments, the diacetyl content is increased by a factor of or of about 9. In some embodiments, the diacetyl content is increased by a factor of or of about 10.

In some embodiments, the method of producing a fermented plant-based composition having cream flavor includes increasing the diacetyl content by a factor described herein.

In some embodiments, the diacetyl content is increased to or to at least 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 20 ppm or more. In some embodiments, the diacetyl content is increased to or to at least 1.5 ppm. In some embodiments, the diacetyl content is increased to or to at least 2 ppm. In some embodiments, the diacetyl content is increased to or to at least 3 ppm. In some embodiments, the diacetyl content is increased to or to at least 4 ppm. In some embodiments, the diacetyl content is increased to or to at least 5 ppm. In some embodiments, the diacetyl content is increased to or to at least 6 ppm. In some embodiments, the diacetyl content is increased to or to at least 7 ppm. In some embodiments, the diacetyl content is increased to or to at least 8 ppm. In some embodiments, the diacetyl content is increased to or to at least 9 ppm. In some embodiments, the diacetyl content is increased to or to at least 10 ppm. In some embodiments, the diacetyl content is increased to or to at least 15 ppm. In some embodiments, the diacetyl content is increased to or to at least 20 ppm.

In some embodiments, the diacetyl content is increased to between or between about 1 ppm to 20 ppm, 1 ppm to 15 ppm, 1 ppm to 14 ppm, 1 ppm to 13 ppm, 1 ppm to 12 ppm, 1 ppm to 11 ppm, 1 ppm to 10 ppm, 1 ppm to 9 ppm, 1 ppm to 8 ppm, 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content is increased to between or between about 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content is increased to or to about 1 ppm to 7 ppm. In some embodiments, the diacetyl content is increased to between or between about 1.5 ppm to 7 ppm. In some embodiments, the diacetyl content is increased to or to about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, or about 15 ppm.

In some embodiments, the methods of producing a fermented plant-based composition having cream flavor produce a diacetyl content of or of at least 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 20 ppm, or more. In some embodiments, the diacetyl content produced is or is at least 1.5 ppm. In some embodiments, the diacetyl content produced is or is at least 2 ppm. In some embodiments, the diacetyl content produced is or is at least 3 ppm. In some embodiments, the diacetyl content produced is or is at least 4 ppm. In some embodiments, the diacetyl content produced is or is at least 5 ppm. In some embodiments, the diacetyl content produced is or is at least 6 ppm. In some embodiments, the diacetyl content produced is or is at least 7 ppm. In some embodiments, the diacetyl content produced is or is at least 8 ppm. In some embodiments, the diacetyl content produced is or is at least 9 ppm. In some embodiments, the diacetyl content is produced is or is at least 10 ppm. In some embodiments, the diacetyl content is produced is or is at least 15 ppm. In some embodiments, the diacetyl content is produced is or is at least 20 ppm. In some embodiments, the diacetyl content produced is in the range of or of about 1 ppm to 20 ppm, 1 ppm to 15 ppm, 1 ppm to 14 ppm, 1 ppm to 13 ppm, 1 ppm to 12 ppm, 1 ppm to 11 ppm, 1 ppm to 10 ppm, 1 ppm to 9 ppm, 1 ppm to 8 ppm, 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content produced is in the range of or of about 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content produced is in the range of or of about 1 ppm to 7 ppm. In some embodiments, the diacetyl content produced is in the range of or of about 1.5 ppm to 7 ppm. In some embodiments, the diacetyl content produced is or is about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, about 15 ppm, or about 20 ppm.

In some embodiments, the method of producing a fermented plant-based composition having cream flavor includes increasing the diacetyl content to an amount described herein.

In some embodiments, the method of producing a fermented plant-based composition having cream flavor includes decreasing the acetaldehyde content by a factor described herein and increasing the diacetyl content by a factor described herein. In some embodiments, the method of producing a fermented plant-based composition having cream flavor includes decreasing the acetaldehyde content by an amount described herein and increasing the diacetyl content by an amount described herein. In some embodiments, the method of producing a fermented plant-based composition having cream flavor includes decreasing the acetaldehyde content to an amount described herein and increasing the diacetyl content to an amount described herein.

In some embodiments, the methods of producing a fermented plant-based composition having cream flavor, e.g., increased cream flavor, result in a ratio of acetaldehyde to diacetyl that is associated with cream flavor, e.g., increased cream flavor. In some embodiments, the ratio of acetaldehyde to diacetyl in the fermented plant-based composition associated with cream flavor, e.g., increased cream flavor, is less than 1, e.g., about 0.9, about 0.8, about 0.7, about 0.6, about 0.5, about 0.4, about 0.3, about 0.2, about 0.1, about 0.08, about 0.07, about 0.06, about 0.05, about 0.04, about 0.03, about 0.02, or about 0.01, or less. In some embodiments, the ratio of acetaldehyde to diacetyl in the fermented plant-based composition associated with cream flavor, e.g., increased cream flavor, is less than or less than about 0.85, 0.75, 0.5, 0.25, 0.1, 0.075, 0.05, 0.025, or 0.01. In some embodiments, the ratio of acetaldehyde diacetyl in the fermented plant-based composition associated with cream flavor, e.g., increased cream flavor, is less than or less than about 0.7, 0.5, 0.4, 0.3, 0.2, or 0.1. In some embodiments, the ratio of acetaldehyde diacetyl in the fermented plant-based composition associated with cream flavor, e.g., increased cream flavor, is or is about 0.6. In some embodiments, the ratio of acetaldehyde diacetyl in the fermented plant-based composition associated with cream flavor, e.g., increased cream flavor, is less than or less than about 0.25, 0.1, 0.075, 0.05, 0.025, or 0.01. In some embodiments, the ratio of acetaldehyde to diacetyl in the fermented plant-based compositions associated with cream flavor, e.g., increased cream flavor, is in the range of or of about 0.8 to 0, 0.7 to 0, 0.6 to 0, 0.5 to 0, 0.4 to 0, 0.3 to 0, 0.2 to 0, or 0.1 to 0. In some embodiments, the ratio of acetaldehyde to diacetyl in the fermented plant-based composition associated with cream flavor, e.g., increased cream flavor, is in the range of or of about 0.6 to 0. In some embodiments, the ratio embodiments, the ratio of acetaldehyde to diacetyl in the fermented plant-based compositions associated with cream flavor, e.g., increased cream flavor, is or is about 0, e.g., when the acetaldehyde content is at or below the lower limit of detection and/or quantification.

In some embodiments, the acetaldehyde content is decreased to produce a ratio of acetaldehyde to diacetyl that is less than 0.8, 0.7, 0.6, 0.5, 0.4, 0.3, 0.2, 0.1, 0.09, 0.08, 0.07, 0.06, 0.05, 0.04, 0.03, 0.02, or 0.01, or 0.

In some embodiments, the acetaldehyde content is decreased and the diacetyl content is increased to produce a ratio of acetaldehyde to diacetyl that is or is less than 0.8, 0.7, 0.6, 0.5, 0.4, 0.3, 0.2, 0.1, 0.09, 0.08, 0.07, 0.06, 0.05, 0.04, 0.03, 0.02, or 0.01, or 0. In some embodiments, the acetaldehyde content is decreased and the diacetyl content is increased to produce a ratio of acetaldehyde to diacetyl that is or is less than 0.8, 0.7, 0.6, 0.5, 0.4, 0.3, 0.2, 0.1, 0.09, 0.08, 0.07, 0.06, 0.05, 0.04, 0.03, 0.02, or 0.01, or 0 and the diacetyl content is increased to at least or at least about 1 ppm. In some embodiments, the acetaldehyde content is decreased and the diacetyl content is increased to produce a ratio of acetaldehyde to diacetyl that is or is less than 0.7, 0.6, 0.5, 0.4, 0.3, 0.2, 0.1 or is or is about 0, and the diacetyl content is increased to at least or at least about 1 ppm. In some embodiments, the acetaldehyde content is decreased and the diacetyl content is increased to produce a ratio of acetaldehyde to diacetyl that is or is less than 0.8, 0.7, 0.6, 0.5, 0.4, 0.3, 0.2, 0.1, 0.08, 0.07, 0.06, 0.05, 0.04, 0.03, 0.02, or 0.01, or 0, and the diacetyl content is increased to at least or at least about 1.5 ppm. In some embodiments, the acetaldehyde content is decreased and the diacetyl content is increased to produce a ratio of acetaldehyde to diacetyl that is or is less than 0.7, 0.6, 0.5, 0.4, 0.3, 0.2, 0.1 or is or is about 0, and the diacetyl content is increased to at least or at least about 1.5 ppm. In some embodiments, the acetaldehyde content is decreased and the diacetyl content is increased to produce a ratio of acetaldehyde to diacetyl that is or is less than 0.6, 0.5, 0.25, 0.1 or is or is about 0, and the diacetyl content is increased to a level disclosed in any embodiment herein.

In some embodiments, the diacetyl content is increased to about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, about 15 ppm, or about 20 ppm and the acetaldehyde content is decreased to produce a ratio of acetaldehyde to diacetyl that is or is less than 0.8, 0.7, 0.6, 0.5, 0.4, 0.3, 0.2, 0.1, 0.09, 0.08, 0.07, 0.06, 0.05, 0.04, 0.03, 0.02, 0.01, or 0. In some embodiments, the diacetyl content is increased to about 1 ppm, about 2 ppm, about 3 ppm, about 4 ppm, about 5 ppm, about 6 ppm, about 7 ppm, and the acetaldehyde content is decreased to produce a ratio of acetaldehyde to diacetyl that is or is less than 0.7, 0.6, 0.5, 0.4, 0.3, 0.2, 0.1, or 0. In some embodiments, the diacetyl content is increased to about 1 ppm, about 2 ppm, about 3 ppm, about 4 ppm, about 5 ppm, about 6 ppm, about 7 ppm, and the acetaldehyde content is decreased to produce a ratio of acetaldehyde to diacetyl that is or is less than 0.7, 0.6. 0.5, 0.4, 0.3, 0.2, 0.1, or 0. In some embodiments, the acetaldehyde content is decreased to a level disclosed herein to achieve a ratio associated with cream flavor, e.g., increased cream flavor, disclosed herein.

In some embodiments, the acetaldehyde content is decreased to below about 1 ppm, and the diacetyl content is increased to above or above about 1 ppm. In some embodiments, the acetaldehyde content is decreased to below about 0.7 ppm, and the diacetyl content is increased to above about 1 ppm. In some embodiments, the acetaldehyde content is decreased to about 0.6 ppm and the diacetyl content is increased to above or above about 1.5 ppm. In some embodiments, the acetaldehyde content is decreased to below about 1 ppm, and the diacetyl content is increased to above or above about 1.5 ppm. In some embodiments, the acetaldehyde content is decreased to below about 0.5 ppm, and the diacetyl content is increased to above about 1.5 ppm. In some embodiments, the acetaldehyde content is decreased to about 0 ppm and the diacetyl content is increased to above or above about 1.5 ppm. In some embodiments, the acetaldehyde content is decreased to any level disclosed herein and the diacetyl content is increased to any level disclosed herein to obtain a ratio of acetaldehyde to diacetyl disclosed herein, e.g., a ratio correlated with cream flavor, e.g., increased cream flavor.

In some embodiments the methods of producing a fermented plant-based composition having cream flavor, e.g., increased cream flavor, produces an acetaldehyde content that is below about 1 ppm, and a diacetyl content is above or above about 1 ppm. In some embodiments, the acetaldehyde content produced is below about 0.6 ppm, and the diacetyl content is produced is above about 1 ppm. In some embodiments, the acetaldehyde content produced is about 0.6 ppm and the diacetyl content produced is above or above about 1.5 ppm. In some embodiments, the acetaldehyde content produced is any level disclosed herein and the diacetyl content produced is any level disclosed herein to obtain a ratio of acetaldehyde to diacetyl disclosed herein, e.g., a ratio correlated with cream flavor, e.g., increased cream flavor.

In some embodiments, the cream flavor of the fermented plant-based compositions having levels of acetaldehyde and diacetyl and/or a ratio thereof described herein has an increased cream aroma and/or increased cream odor compared to a fermented plant-based compositions having an acetaldehyde content and a diacetyl content resulting in a ratio of acetaldehyde to diacetyl of about equal to or greater than 0.7. In some embodiments, the cream flavor of the fermented plant-based compositions having levels of acetaldehyde and diacetyl or a ratio thereof described herein has an increased cream aroma and/or increased cream odor compared to a fermented plant-based compositions having an acetaldehyde content and a diacetyl content resulting in a ratio of acetaldehyde to diacetyl of about equal to or greater than 0.8. In some embodiments, the cream flavor of the fermented plant-based compositions having levels of acetaldehyde and diacetyl or a ratio thereof described herein has an increased cream aroma and/or increased cream odor compared to a fermented plant-based compositions having an acetaldehyde content and a diacetyl content resulting in a ratio of acetaldehyde to diacetyl of about equal to or greater than 0.9. In some embodiments, the cream flavor of the fermented plant-based compositions having levels of acetaldehyde and diacetyl or a ratio thereof described herein has an increased cream aroma and/or increased cream odor compared to a fermented plant-based compositions having an acetaldehyde content and a diacetyl content resulting in a ratio of acetaldehyde to diacetyl of about equal to or greater than 1.

In some embodiments, the content or ratio described in this Section that associates with a cream flavor, e.g., increased cream flavor, is achieved at the end of fermentation. In some embodiments, the content or ratio described in this Section that associates with a cream flavor, e.g., increased cream flavor, is achieved at a time point after the end of fermentation. For example, the content or ratio described in this Section that associates with a cream flavor, e.g., increased cream flavor, is achieved during storage of the fermented plant-based composition. In some embodiments, the content or ratio described in this Section, which correlates with a cream flavor, e.g., increased cream flavor, is achieved in a stored fermented plant-based composition. For example, in some cases, the content or ratio described in this Section, which correlates with a cream flavor, e.g., increased cream flavor, is achieved during storage of the fermented plant-based composition, e.g., stored fermented plant-based composition according to any of the methods of storage described in Section I-D above.

In some embodiments, the content or ratio described in this Section of acetaldehyde to diacetyl associated with a cream flavor, e.g., increased cream flavor, is reached after more than 1 day of storage of the fermented plant-based composition. In some embodiments, content or ratio described in this Section of acetaldehyde to diacetyl associated with a cream flavor, e.g., increased cream flavor, is reached in or in about a range of 1 to 30, 1 to 25, 1 to 20, 1 to 15, 1 to 10, or 1 to 5 days of storage of the fermented plant-based composition. In some embodiments, the content or ratio described in this Section of acetaldehyde to diacetyl associated with a cream flavor, e.g., increased cream flavor, is reached in or in about a range of 1 to 28, 1 to 21, or 1 to 14 or 1 to 7 days of storage of the fermented plant-based composition.

In some embodiments, the content or ratio described in this Section of acetaldehyde to diacetyl associated with a cream flavor, e.g., increased cream flavor, is reached within or less than 28 days of storage of the fermented plant-based composition. In some embodiments, the content or ratio described in this Section of acetaldehyde to diacetyl associated with a cream flavor, e.g., increased cream flavor, is reached within or less than 21 days of storage of the fermented plant-based composition. In some embodiments, the content or ratio described in this Section of acetaldehyde to diacetyl associated with a cream flavor, e.g., increased cream flavor, is reached within or less than 14 days of storage of the fermented plant-based compositions. In some embodiments, the content or ratio described in this Section of acetaldehyde to diacetyl associated with a cream flavor, e.g., increased cream flavor, is reached within or less than 7 days of storage of the fermented plant-based compositions.

In some embodiments, the content or ratio described in this Section of acetaldehyde to diacetyl associated with a cream flavor, e.g., increased cream flavor, is reached in or in about 28 days of storage of the fermented plant-based compositions. In some embodiments, the content or ratio described in this Section of acetaldehyde to diacetyl associated with a cream flavor, e.g., increased cream flavor, is reached in or in about 21 days of storage of the fermented plant-based compositions. In some embodiments, the content or ratio described in this Section of acetaldehyde to diacetyl associated with a cream flavor, e.g., increased cream flavor, is reached in or in about than 14 days of storage of the fermented plant-based compositions. In some embodiments, the content or ratio described in this Section of acetaldehyde to diacetyl associated with a cream flavor, e.g., increased cream flavor, is reached in or in about 7 days of storage of the fermented plant-based compositions.

### F. Fermented Plant-Based Food Products

Also provided herein are fermented plant-based food products obtained by any of the methods described herein and having any of the properties, e.g., ratios, described herein that produce a cream flavor, such as an increased cream flavor. In some aspects, a fermented plant-based composition is a fermented plant-based food product.

In some embodiments, the methods provided herein to produce a fermented plant-based composition having cream flavor produce a fermented plant-based food product. In some embodiments, the fermented plant-based food product contains a ratio of acetaldehyde content to diacetyl content that is less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01, or is 0. In some embodiments, the fermented plant-based food product contains a ratio of acetaldehyde content to diacetyl content that is less than 0.7, 0.6, 0.5, 0.25, 0.1, 0.075, 0.05, 0.025, or 0.01, or is 0. In some embodiments, the fermented plant-based food product contains a ratio of acetaldehyde content to diacetyl content that is less than 0.7, 0.6, 0.5, 0.4, 0.3, 0.2, 0.1 or is 0. In some embodiments, the fermented plant-based food product contains a ratio of acetaldehyde content to diacetyl content that is or is less than 0.7. In some embodiments, the fermented plant-based food product contains a ratio of acetaldehyde content to diacetyl content that is or is less than 0.6. In some embodiments, the content or ratio of acetaldehyde to diacetyl in the fermented plant-based food product is any as described in Section I-E associated with cream flavor, e.g., an increased cream flavor.

In some embodiments, the fermented plant-based food product contains a Lacticaseibacillus rhamnosus strain that decreases acetaldehyde in the fermented plant-based food product. In some embodiments, the fermented plant-based food product contains a Lacticaseibacillus rhamnosus strain that increases diacetyl in the fermented plant-based food product. In some embodiments, the Lacticaseibacillus rhamnosus strain that decreases acetaldehyde and increases diacetyl is the same strain. Alternatively, different Lacticaseibacillus rhamnosus strains may decrease acetaldehyde and increase diacetyl.

In some embodiments, the fermented plant-based food product contains a Lacticaseibacillus rhamnosus strain DSM33650 or a mutant thereof as described herein. In some embodiments, the fermented plant-based food product contains a Lacticaseibacillus rhamnosus strain DSM22876 or a mutant thereof as described herein. In some embodiments, the fermented plant-based food product contains a Lacticaseibacillus rhamnosus strain DSM33850 or a mutant thereof as described herein. In some embodiments, the fermented plant-based food product contains a Streptococcus thermophilus.

In some embodiments, the fermented plant-based food product contains Streptococcus thermophilus strain DSM33651 or a mutant thereof as described herein and a Lacticaseibacillus rhamnosus strain DSM33650 or a mutant thereof as described herein. In some embodiments, the fermented plant-based food product contains Streptococcus thermophilus strain DSM33651 or a mutant thereof as described herein and a Lacticaseibacillus rhamnosus strain DSM22876 or a mutant thereof as described herein. In some embodiments, the fermented plant-based food product contains Streptococcus thermophilus strain DSM33651 or a mutant thereof as described herein and a Lacticaseibacillus rhamnosus strain DSM33850 or a mutant thereof as described herein.

In some embodiments, the fermented plant-based food product is a soy-based composition. In some embodiments, the fermented plant-based food product is a fermented soy-based food product.

Fermented plant-based food products contemplated herein include, but are not limited to, a food such as, but not limited to, fermented dairy alternative food products. In some embodiments, the fermented plant-based food product is a fermented dairy alternative food product.

The term “food” is used in a broad sense and includes feeds, foodstuffs, food ingredients, food supplements, and functional foods. Here, the term “food” is used in a broad sense—and covers food for humans as well as food for animals (i.e. a feed). In a preferred aspect, the food is for human consumption.

In some embodiments, the fermented plant-based composition produced according to the methods described herein produces a fermented plant-based food product that is a functional food. As used herein, the term “functional food” means a food which is capable of providing not only a nutritional effect and/or a taste satisfaction, but is also capable of delivering a further beneficial effect to consumer. Although there is no legal definition of a functional food, most of the parties with an interest in this area agree that there are foods marketed as having specific health effects.

In some embodiments, the bacterial strains or bacterial compositions, e.g., of the starter culture, described herein may be—or may be added to—a food ingredient, a food supplement, or a functional food.

In some embodiments, the food may be in the form of a solution or as a solid—depending on the use and/or the mode of application and/or the mode of administration.

In some embodiments, the bacterial strains or bacterial compositions, e.g., start cultures, described herein can be used in the preparation of food products such as one or more of fermented plant-based food products.

By way of example, the bacterial strain or bacterial composition, e.g., starter culture, can be used as an ingredient to prepare milk-type drinks, lactic acid bacteria drinks, yoghurt alternatives, and/or drinking yoghurt alternatives that are made from plant matter (e.g., a vegetal base) and referred to as a dairy alternative food product, e.g., plant-based yoghurt alternative.

In some embodiments, the fermented plant-based food product is a stirred type dairy alternative food product. In some embodiments, the fermented plant-based food product is a set-type dairy alternative food product. In some embodiments, the fermented pant-based food product is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.

### II. METHODS OF IDENTIFYING BACTERIA CAPABLE OF PRODUCING FERMENTED PLANT-BASED COMPOSITIONS HAVING CREAM FLAVOR

Provided herein are methods of identifying bacteria for producing fermented plant-based compositions, e.g., fermented plant-based food products, having cream flavor, e.g., increased cream flavor. In some embodiments, the method for identifying bacteria and bacterial compositions capable of producing fermented plant-based compositions having cream flavor, e.g., increased cream flavor, includes fermenting a vegetal base as described herein inoculated with one or more bacteria and determining whether the one or more bacteria produce increases in diacetyl and/or decreases in acetaldehyde content in the fermented plant-based composition that produce ratios and/or levels of acetaldehyde and diacetyl that are associated, e.g., correlated, with cream flavor, e.g., increased cream flavor. In some cases, the one or more bacteria able to produce such a result are identified as capable of producing fermented plant-based composition having cream flavor, e.g., increased cream flavor. In some aspects, the methods of identifying such bacteria include quantifying acetaldehyde and diacetyl content in a fermented plant-based composition fermented in the presence of one or more bacteria. In some embodiments the fermented plant-based composition is produced, e.g., fermented and stored, according the methods provided herein, with bacteria or bacterial compositions to be assessed for their ability to produce levels and ratios of acetaldehyde and diacetyl associated with cream flavor. In some embodiments, one or more bacteria capable of producing a fermented plant-based composition having cream flavor are identified.

In some embodiments, the method of identifying bacteria capable of producing fermented plant-based compositions having cream flavor as described herein includes determining the ratio of acetaldehyde to diacetyl in a fermented plant-based composition fermented in the presence of one or more bacteria. In some embodiments, the method of identifying includes quantifying acetaldehyde content and diacetyl content in a fermented plant-based composition fermented in the presence of one or more bacteria and stored following fermentation, for example as described herein. In some embodiments, the method of identifying includes determining the ratio of acetaldehyde to diacetyl in a fermented plant-based composition fermented in the presence of one or more bacteria and stored following fermentation as described herein. In some embodiments, the method of identifying includes quantifying acetaldehyde content and diacetyl content in a stored fermented plant-based composition fermented in the presence of one or more bacteria. In some embodiments, the method of identifying includes determining the ratio of acetaldehyde to diacetyl in a stored fermented plant-based composition, as described herein, fermented in the presence of one or more bacteria. In some embodiments, the quantifying of content and/or determination of ratio as described herein takes place at different time points over the duration of storage. Thus, in some cases, the change in diacetyl and/or acetaldehyde content may be determined over time. In some embodiments, the quantifying of content and/or determination of ratio as described herein takes place continuously over the duration of storage.

In some embodiments, if the one or more bacteria produce a decrease in acetaldehyde levels as described in Section I-E, the one or more bacteria are identified as capable of producing fermented plant-based compositions having cream flavor. In some embodiments, if the one or more bacteria produce a decrease in acetaldehyde and an increase in diacetyl levels as described in Section I-E, the one or more bacteria are identified as capable of producing fermented plant-based compositions having cream flavor. In some embodiments, if the one or more bacteria produce a ratio of acetaldehyde to diacetyl associated with cream flavor as described in Section I-E, the one or more bacteria are identified as capable of producing fermented plant-based compositions having cream flavor. In some embodiments, if the one or more bacteria produce a level and/or ratio of acetaldehyde to diacetyl associated with cream flavor as described in Section I-E, the one or more bacteria are identified as capable of producing fermented plant-based compositions having cream flavor.

In some embodiments, the bacteria identified as capable of producing fermented plant-based compositions having cream flavor produce a decrease in acetaldehyde levels as described in Section I-E. In some embodiments, the bacteria identified as capable of producing fermented plant-based compositions having cream flavor produce a decrease in acetaldehyde and an increase in diacetyl levels as described in Section I-E. In some embodiments, the bacteria identified as capable of producing fermented plant-based compositions having cream flavor produce a ratio of acetaldehyde to diacetyl associated with cream flavor as described in Section I-E. In some embodiments, the bacteria identified as capable of producing fermented plant-based compositions having cream flavor produce a level and/or ratio of acetaldehyde to diacetyl associated with cream flavor as described in Section I-E.

In some embodiments, the bacteria identified as capable of producing fermented plant-based compositions having cream flavor is or includes Lacticaseibacillus rhamnosus strains. In some embodiments, the Lacticaseibacillus rhamnosus strains thereof are of vegetal origin. In some embodiments, the bacteria identified as capable of producing fermented plant-based compositions having cream flavor is or includes Streptococcus thermophilus strains. In some embodiments, the bacteria identified as capable of producing fermented plant-based compositions having cream flavor is or includes Lacticaseibacillus rhamnosus strains and Streptococcus thermophilus strains.

In some aspects, one or more bacteria identified as capable of producing a fermented plant-based composition having cream flavor are contained in a composition. In some embodiments, the composition is a starter culture. In some embodiments, the starter culture further includes one or more bacteria described in Section I-B. In some embodiments, the composition is a fermented plant-based food product. In some embodiments, the fermented plant-based food product is any one of those described in Section I-F.

### III. KITS

Also provided are kits including the compositions containing bacteria and bacterial compositions, e.g., starter cultures, described herein, which may further include instructions for using the compositions, such as described herein. The kits described herein may also include other materials desirable from a commercial and user standpoint, including other buffers, diluents, filters, needles, syringes, and package inserts with instructions for performing any methods described herein. In some embodiments, the kits provided herein include bacteria and/or bacterial compositions capable of producing a fermented plant-based composition having cream flavor, such as an increased cream flavor.

### Deposit and Expert Solution

The following deposit was made according to the Budapest treaty on the international recognition of the deposit of microorganisms for the purposes of patent procedure.
Lacticaseibacillus rhamnosus strain (DGCC1179) deposited under accession number DSM33650 on Sep. 29, 2020, at the DSMZ [Deutsche Sammlung von Mikroorganismen und Zellkulturen GmbH, Inhoffenstrasse 7B, D-38124 Braunschweig-Germany].
Lacticaseibacillus rhamnosus strain (DGCC1460) deposited under accession number DSM22876 on Jun. 1, 2021, at the DSMZ [Deutsche Sammlung von Mikroorganismen und Zellkulturen GmbH, Inhoffenstrasse 7B, D-38124 Braunschweig-Germany].
Lacticaseibacillus rhamnosus strain (DGCC13617) deposited under accession number DSM33850 on Apr. 20, 2021, at the DSMZ [Deutsche Sammlung von Mikroorganismen und Zellkulturen GmbH, Inhoffenstrasse 7B, D-38124 Braunschweig-Germany].
Streptococcus thermophilus strain (DGCC11042) deposited under accession number DSM 33651 on Sep. 29, 2020, at the DSMZ [Deutsche Sammlung von Mikroorganismen und Zellkulturen GmbH, Inhoffenstrasse 7B, D-38124 Braunschweig-Germany].

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC)

### IV. EXEMPLARY EMBODIMENTS

Among the provided embodiments are:
1. A method for producing a fermented plant-based composition having a cream flavor, the method comprising decreasing acetaldehyde content in the fermented plant-based composition.
2. A method for producing a stored fermented plant-based composition having a cream flavor, the method comprising:
   (a) storing a fermented plant-based composition to produce a stored fermented plant-based composition; and
   (b) decreasing acetaldehyde content in the fermented plant-based composition during storage.
3. The method according to embodiment 1 or embodiment 2, wherein the method further comprises producing the fermented plant-based composition, the producing comprising fermenting a vegetal base inoculated with a starter culture.
4. A method for producing a fermented plant-based composition having a cream flavor, the method comprising:
   (a) fermenting a vegetal base inoculated with a starter culture to produce a fermented plant-based composition; and
   (b) decreasing acetaldehyde content in the fermented plant-based composition.
5. The method of any one of embodiments 1-4, further comprising increasing diacetyl content in the fermented plant-based composition.
6. The method of any one of embodiments 3-5, wherein the vegetal base comprises soy plant matter, optionally in an aqueous suspension.
7. The method of any one of embodiments 1 and 3-6, comprising storing the fermented plant-based composition to produce a stored fermented plant-based composition.
8. The method of embodiment 7, wherein the acetaldehyde content is decreased in the fermented plant-based composition during storage.
9. The method of embodiment 7 or embodiment 8, comprising increasing diacetyl content in the fermented plant-based composition during storage.
10. The method of any one of embodiments 1-9, wherein the acetaldehyde content is decreased to produce a ratio of acetaldehyde to diacetyl that is less than 0.7, 0.6, 0.5, 0.4, 0.3, 0.2, 0.1 or is 0.
11. The method of any one of embodiments 5-10, wherein the acetaldehyde content is decreased and the diacetyl content is increased to produce a ratio of acetaldehyde to diacetyl that is less than 0.7, 0.6, 0.5, 0.4, 0.3, 0.2, 0.1 or is 0.
12. The method of any one of embodiments 3-11, wherein the starter culture comprises one or more lactic acid bacteria, optionally at least two lactic acid bacteria.
13. The method of any one of embodiments 3-12, wherein the starter culture comprises bacteria from the genus Lactococcus, Lactobacillus, Streptococcus, Lacticaseibacillus, Leuconostoc, Pediococcus, or Bifidobacterium, or any combination of the foregoing.
14. The method of any one of embodiments 3-13, wherein the starter culture comprises one or more Lacticaseibacillus rhamnosus strains.
15. The method of embodiment 14, wherein the one or more Lacticaseibacillus rhamnosus strains comprise or consist of:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; and/or
   (c) the Lacticaseibacillus rhamnosus strain deposited under accession number DSM33850 at the DSMZ or a mutant thereof.
16. The method of any one of embodiments 3-15, wherein the starter culture comprises one or more Streptococcus thermophilus strains.
17. The method of embodiment 16, wherein the one or more Streptococcus thermophilus strains comprise or consist of the Streptococcus thermophilus strain deposited under accession number DSM33651 at the DSMZ or a mutant thereof.
18. The method of any one of embodiments 3-17, wherein the starter culture comprises or consists of:
   (a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof;
   (b) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; or
   (c) the Lacticaseibacillus rhamnosus strain deposited under accession number DSM33850 at the DSMZ or a mutant thereof and the Streptococcus thermophilus strain deposited under accession number DSM33651 at the DSMZ or a mutant thereof.
19. The method of any one of embodiments 3-18, wherein the starter culture comprises one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus cremoris strain, a Lactococcus lactis subsp lactis strain, or a Lactococcus cremoris subsp cremoris strain.
20. The method of any one of embodiments 3-19, wherein the inoculated vegetal base is fermented at a temperature above about 33° C., 35° C., 36° C., 37° C., 38° C., 39° C., 40° C., 41° C., 42° C., 43° C., 44° C., 45° C., or 50° C.
21 The method of any one of embodiments 2, 3, and 7-20, wherein the fermented plant-based composition is stored at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or about 26° C.
22. The method of any one of embodiments 2, 3, and 7-21, wherein the fermented plant-based composition is stored for:
   (a) at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days; or
   (b) at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days.
23. The method of any one of embodiments 2, 3, and 7-22, wherein the fermented plant-based composition is stored for about 28 days at about 6° C.
24 The method of any one of embodiments 2, 3, and 7-22, wherein the fermented plant-based composition is stored for about 14 days at about 6° C.
25. The method of any one of embodiments 2, 3, and 7-22, wherein the fermented plant-based composition is stored for about 7 days at about 6° C.
26. The method of any one of embodiments 1-25, wherein the cream flavor comprises a cream aroma and/or a cream odor.
27. The method of any of embodiments 1-26, wherein the cream flavor is an increased cream flavor compared to a cream flavor of a fermented plant-based composition having an acetaldehyde content and a diacetyl content resulting in a ratio of acetaldehyde to diacetyl of equal to or greater than 0.7.
28 The method of embodiment 27, wherein the increased cream flavor comprises an increased cream aroma and/or cream odor compared to a cream aroma and/or cream odor of a fermented plant-based composition having an acetaldehyde content and a diacetyl content resulting in a ratio of acetaldehyde to diacetyl of equal to or greater than 0.7.
29. A composition comprising a Lacticaseibacillus rhamnosus strain, wherein the Lacticaseibacillus rhamnosus strain decreases acetaldehyde in a fermented plant-based composition to produce a cream flavor.
30 The composition of embodiment 29, wherein the Lacticaseibacillus rhamnosus strain is of vegetal origin.
31 The composition of embodiment 29 or embodiment 30, wherein the Lacticaseibacillus rhamnosus strain is:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; or
   (c) the Lacticaseibacillus rhamnosus strain deposited under accession number DSM33850 at the DSMZ or a mutant thereof.
32 The composition of any one of embodiments 29-31, further comprising a Streptococcus thermophilus strain.
33. The composition of embodiment 32, wherein the Streptococcus thermophilus strain is the Streptococcus thermophilus strain deposited under accession number DSM33651 at the DSMZ or a mutant thereof.
34. The composition of embodiment 32 or embodiment 33, comprising or consisting of:
   (a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof;
   (b) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; or
   (c) the Lacticaseibacillus rhamnosus strain deposited under accession number DSM33850 at the DSMZ or a mutant thereof and the Streptococcus thermophilus strain deposited under accession number DSM33651 at the DSMZ or a mutant thereof.
35. The composition of any one of embodiments 29-34, further comprising one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus cremoris subsp cremoris, or a Lactococcus lactis subsp lactis strain.
36. The composition of any one of embodiments 29-35, wherein the composition is a starter culture or a fermented plant-based food product.
37. Use of a Lacticaseibacillus rhamnosus to decrease acetaldehyde content in a fermented plant-based composition to produce a cream flavor.
38. The use of embodiment 37, wherein the Lacticaseibacillus rhamnosus strain is:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; or
   (c) the Lacticaseibacillus rhamnosus strain deposited under accession number DSM33850 at the DSMZ or a mutant thereof.
39. The composition of any one of embodiments 29-36 or the use of embodiment 37 or embodiment 38, wherein the Lacticaseibacillus rhamnosus increases diacetyl content in the fermented plant-based composition.
40. The composition of any one of embodiments 29-36 and 39 or the use of any one of embodiments 37-39, wherein the acetaldehyde content is decreased to produce a ratio of acetaldehyde to diacetyl that is less than 0.7, 0.6, 0.5, 0.4, 0.3, 0.2, 0.1 or is 0.
41. The composition of embodiment 39 or embodiment 40 or the use of embodiment 39 or embodiment 40, wherein the acetaldehyde content is decreased and the diacetyl content is increased to produce a ratio of acetaldehyde to diacetyl that is less than 0.7, 0.6, 0.5, 0.4, 0.3, 0.2, 0.1 or is 0.
42. The composition of any one of embodiments 29-36 and 39-41 or the use of any one of embodiments 37-41, wherein the cream flavor comprises a cream aroma and/or a cream odor.
43. The composition of any one of embodiments 29-36 and 39-42 or the use of any one of embodiments 37-42, wherein the cream flavor is an increased cream flavor compared to a cream flavor of a fermented plant-based composition having an acetaldehyde content and a diacetyl content resulting in a ratio of acetaldehyde to diacetyl of equal to or greater than 0.7.
44. The composition of embodiment 43 or the use of embodiment 43, wherein the increased cream flavor comprises an increased cream aroma and/or cream odor compared to a cream aroma and/or cream odor of a fermented plant-based composition having an acetaldehyde content and a diacetyl content resulting in a ratio of acetaldehyde to diacetyl of equal to or greater than 0.7.
45. A fermented plant-based composition obtained by the method of any one of embodiments 1-28 or the use of any one of embodiments 37-44.
46. The fermented plant-based composition of embodiment 45, wherein the fermented plant-based composition is a fermented plant-based food product, optionally a fermented plant-based dairy alternative food product.
47. The fermented plant-based composition of embodiment 45 or embodiment 46, wherein the fermented plant-based composition is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.
48. A fermented plant-based food product having cream flavor, the fermented plant-based food product comprising a ratio of acetaldehyde content to diacetyl content that is less than 0.7, 0.6, 0.5, 0.4, 0.3, 0.2, 0.1 or is 0.
49. The fermented plant-based food product having cream flavor of embodiment 48, wherein the fermented plant-based food product comprises a Lacticaseibacillus rhamnosus strain that decreases acetaldehyde in the fermented plant-based food product.
50. The fermented plant-based product having cream flavor of embodiment 48 or embodiment 49, wherein the Lacticaseibacillus rhamnosus strain increases diacetyl in the fermented plant-based food product
51. The fermented plant-based product having cream flavor of any one of embodiments 48-50, wherein the Lacticaseibacillus rhamnosus strain is:
   (a) the Lacticaseibacillus rhamnosus strain deposited under accession number DSM33650 or a mutant thereof;
   (b) the Lacticaseibacillus rhamnosus strain deposited under accession number DSM22876 or a mutant thereof; and/or
   (c) the Lacticaseibacillus rhamnosus strain deposited under accession number DSM33850 or a mutant thereof.
52. The fermented plant-based food product having cream flavor of any one of embodiments 48-51, further comprising a Streptococcus thermophilus strain.
53. The fermented plant-based food product having cream flavor embodiment 52, wherein the Streptococcus thermophilus strain is the Streptococcus thermophilus strain deposited under accession number DSM33651 or a mutant thereof.
54. The fermented plant-based food product having cream flavor of any one of embodiments 48-53, wherein the fermented plant-based food product is a fermented plant-based dairy alternative food product.
55. The fermented plant-based food product of any one of embodiments 48-54, wherein the fermented plant-based food product is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.
56. A method of identifying bacteria capable of producing a fermented plant-based composition having cream flavor, the method comprising quantifying acetaldehyde content and diacetyl content in a fermented plant-based composition fermented in the presence of one or more bacteria.
57. The method of embodiment 56, wherein the fermented plant-based composition is stored and the acetaldehyde content and diacetyl content are quantified during storage.
58. The method of embodiment 56 or embodiment 57, wherein if the quantified acetaldehyde content and diacetyl content produce a ratio of acetaldehyde content to diacetyl content that is less than 0.7, 0.6, 0.5, 0.4, 0.3, 0.2, 0.1 or is 0, the one or more bacteria are identified as capable of producing a fermented plant-based composition having cream flavor.
59. The method according to any one of embodiments 56-58, wherein the one or more bacteria comprise lactic acid bacteria.
60. The method according to any one of embodiments 56-59, wherein the one or more bacteria comprise a Lacticaseibacillus rhamnosus strain, optionally wherein the Lacticaseibacillus rhamnosus strain is of vegetal origin.
61. The method according to any one of embodiments 56-60, wherein the one or more bacteria comprise a Streptococcus thermophilus strain.
62 The method of any one of embodiments 10-28 and 56-61, the composition of any one of embodiments 40-44, the use according to any one of embodiments 40-44, the fermented plant-based composition according to any one of embodiments 45-47, or the fermented plant-based food product according to any one of embodiments 48-55, wherein the ratio of acetaldehyde to diacetyl is reached after 28 days or less in storage.
63. The method of any one of embodiments 10-28 and 56-62, the composition of any one of embodiments 40-44 and 62, the use according to any one of embodiments 40-44 and 62, the fermented plant-based composition according to any one of embodiments 45-47 and 62, or the fermented plant-based food product according to any one of embodiments 48-55 and 62, wherein the ratio of acetaldehyde to diacetyl is reached after 21 days or less in storage.
64. The method of any one of embodiments 10-28 and 56-63, the composition of any one of embodiments 40-44, 62, and 63, the use according to any one of embodiments 40-44, 62, and 63, the fermented plant-based composition according to any one of embodiments 45-47, 62, and 63, or the fermented plant-based food product according to any one of embodiments 48-55, 62, and 63, wherein the ratio of acetaldehyde to diacetyl is reached after 14 days or less in storage.
65. The method of any one of embodiments 10-28 and 56-64, the composition of any one of embodiments 40-44 and 62-64, the use according to any one of embodiments 40-44 and 62-64, the fermented plant-based composition according to any one of embodiments 45-47 and 62-64, or the fermented plant-based food product according to any one of embodiments 48-55 and 62-64, wherein the ratio of acetaldehyde to diacetyl is reached after 7 days or less in storage.
66. A composition comprising one or more bacteria identified as capable of producing a fermented plant-based composition having cream flavor according to the method of any one of embodiments 56-65.
67. The composition of embodiment 66, wherein the composition is a starter culture or a fermented plant-based food product, optionally a fermented plant-based dairy alternative food product.
68. A kit comprising:
   (a) the composition of any one of embodiments 29-36, 40-44, and 62-67; and
   (b) instructions for use.
69. A Lacticaseibacillus rhamnosus strain deposited under accession number DSM33850 at the DSMZ or a mutant thereof.
70. A Lacticaseibacillus rhamnosus strain deposited under accession number DSM22876 or a mutant thereof.
71. A Streptococcus thermophilus strain deposited under accession number DSM33651 at the DSMZ or a mutant thereof.

### V. EXAMPLES

The following examples are included for illustrative purposes only and are not intended to limit the scope of the invention.

### Example 1: Production of Volatile Organic Compounds Associated with Cream Flavor in Fermented Plant-Based Yogurt Alternatives

The ability to produce volatile organic compounds acetaldehyde and diacetyl at levels correlated with cream flavor using bacterial compositions was assessed in fermented plant-based yogurt alternative products.

### A. Materials and Methods

### Substrates

An exemplary soy substrate for producing a thermophilic fermented plant-based yogurt alternative was assessed. Table E1 describes the raw material and plant-based substrate used for fermentation.

TABLE E1
Properties of the exemplary soy raw material
and plant-based substrate for fermentation.
Raw materials

| | |
|---|---|
| Raw material | Soy drink no sugar |
| Brand | Bjorg |

Plant-based substrate (approx. % Composition)

| | |
|---|---|
| Fat | 2.3 |
| Sugar (dextrose) | 2 added |
| Proteins | 3.5 |

### Thermophilic Fermentation

The soy plant-based substrate (1 L), as described in Table E1, was inoculated with Streptococcus thermophilus (S. thermophilus) DSM33651 either alone or in combination with one of four exemplary Lacticaseibacillus rhamnosus (L. rhamnosus) strains: DSM33650, DSM33850, DSM22876, or DSM24616. S. thermophilus was inoculated at 2 U/100 L and the L. rhamnosus strains were inoculated at 1×10⁷ CFU/mL (frozen format).

The inoculated substrates were stirred gently and distributed in 8×125 mL yogurt cups. The cups were placed in a 43° C. water bath for fermentation. Once the plant-based substrates reached a pH of 4.6, the yogurt cups were placed into storage at 6° C. for 28 days.

### Volatile Organic Compounds

Volatile organic compound analysis was performed on the fermented plant-based substrates after 1, 7, 14, 21, and 28 days in storage at 6° C.

Volatile organic compounds were analyzed by gas chromatography-mass spectrometry (GC-MS) (ThermoFisher Scientific, USA; TRACE1300 GC coupled with an ISQ mass detector).

Samples were prepared in 10 mL head-space vials by combining 1 g of NaCl, 2 g of fermented substrate, 1 mL of MilliQ cooled water, and 100 μl of internal standard (solution of butylacetate at 25 ppm). Samples were vortexed prior to placement on the refrigerated autosampler of the gas chromatogram.

Volatile organic compounds were extracted from samples after heating (80° C. for 15 min) and shaking, by ITEX (dynamic headspace). The ITEX Trap was then desorbed in the GC-MS injector at 200° C. Molecules were separated on a ZB-Wax (60 m×0.25 mm×0.25 μm) column from Phenomenex with helium as carrier gas at 1 mL/min.

Volatile organic compounds were ionized by electronic impact (70 eV) and detected by a quadrupole mass spectrometer in full scan mode from 30 to 200 amu.

Key molecules of interest, including diacetyl and acetaldehyde, were quantified by external calibration with correction using an internal standard and expressed in ppm. Samples were injected in triplicate.

### B. Results

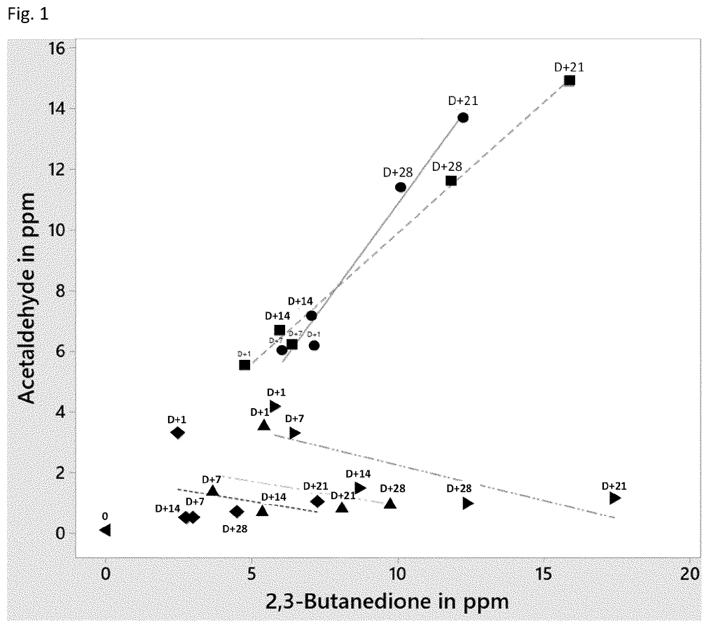

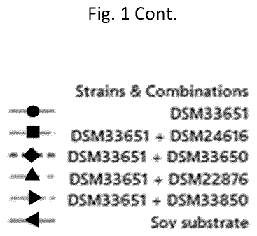

Diacetyl (2,3-butanedione) and acetaldehyde content were detected using GC-MS as described above at different time points during storage of the fermented soy-based thermophilic yogurt alternative. FIG. 1 shows a scatterplot of 2,3-butanedione and acetaldehyde contents (in ppm) after 1, 7, 14, 21 and 28 days at 6° C. for the five soy-based yogurt alternatives. The 2,3-butanedione and acetaldehyde content for the soy substrate prior to inoculation and fermentation is also shown

As shown in FIG. 1, after one day of storage, all yogurt alternatives had a level of acetaldehyde that was similar to the level of 2,3-butanedione measured in the respective yogurt alternative. However, acetaldehyde content was generally higher in yogurt alternatives fermented with S. thermophilus alone or in combination with DSM24616 compared to the other yogurt alternatives.

Between days 1 and 7 in storage, a decrease in acetaldehyde content was observed in yogurt alternatives fermented with S. thermophilus in combination with one of L. rhamnosus strains DSM33650 or DSM22876. For these yogurts, after 7 days, the level of acetaldehyde was generally maintained and an increase in 2,3-butanedione was observed.

At 14, 21, and 28 days in storage, yogurt alternatives fermented with S. thermophilus alone or in combination with one of L. rhamnosus strains DSM24616 or DSM33850 showed similar levels of 2,3-butanedione, which were higher compared to the other soy yogurt alternatives. However, the level of acetaldehyde in the yogurt alterative fermented with a combination of S. thermophilus and DSM33850 was lower than yogurts fermented with S. thermophilus alone or combination with DSM24616. See, FIG. 1.

These results are supportive of the ability of compositions including S. thermophilus and L. rhamnosus that decrease acetaldehyde content, including exemplary strains DSM33650, DSM33850, or DSM22876, to produce in fermented soy-based thermophilic yogurt alternatives levels of acetaldehyde and diacetyl correlated with cream flavor, e.g., cream odor and cream aroma. These results also suggest that the inclusion of L. rhamnosus strains during fermentation can facilitate changes in these volatile compounds to impart cream flavor in fermented soy-based thermophilic yogurt-alternatives.

### Example 2: Production of Volatile Organic Compounds Associated with Cream Flavor in Fermented Plant-Based Yogurt Alternative Produced Using Industrial Starter Cultures

The ability to produce volatile organic compounds acetaldehyde and diacetyl at levels correlated with cream flavor using industrial starter cultures in combination with an exemplary Lacticaseibacillus rhamnosus strain was assessed in fermented plant-based yogurt alternative products.

### A. Materials and Methods

### Substrate

An exemplary soy substrate for producing a thermophilic fermented yogurt alternative was assessed. Table E2 describes the raw materials and plant-based substrates used for fermentation.

TABLE E2
Properties of the exemplary soy raw material
and plant-based substrate for fermentation
Raw material

| | |
|---|---|
| Raw material | Soy drink |
| Brand | Naturli' |

Plant-based substrate (approx. % Composition)

| | |
|---|---|
| Fat | 2.1 |
| Sugar (dextrose) | 2 added |
| Proteins | 3.5 |

### Thermophilic Fermentation

The plant-based substrates were inoculated with one of two industrial starter cultures, each containing Streptococcus thermophilus and Lactobacillus delbrueckii subsp. bulgaricus, either alone or in combination with exemplary Lacticaseibacillus rhamnosus strain DSM33650. The industrial starter cultures were inoculated at 20 DCU/100 L and the L. rhamnosus strain was inoculated at 1×10⁷ CFU/mL.

Inoculated substrates were stirred gently and placed at 40° C. for fermentation. Once the plant-based substrate reached a pH of 4.6, the yogurt cups were placed into storage at 6° C. for 7 days.

Volatile organic compound analysis was performed as described in Example 1 after 7 days in storage at 6° C.

### B. Results

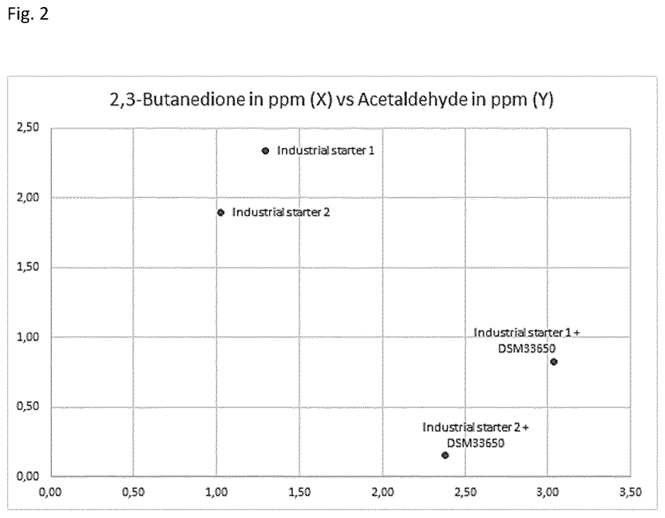

Diacetyl (2,3-butanedione) and acetaldehyde content were detected using GC-MS as described above after 7 days of storage for each of the fermented soy-based thermophilic yogurt alternatives. FIG. 2 shows a scatterplot of 2,3-butanedione and acetaldehyde contents (in ppm) after 7 days of storage at 6° C.

As shown in FIG. 2, after 7 days of storage, acetaldehyde and 2,3-butanedione content were similar for yogurt alternatives fermented with industrial starter cultures in combination with L. rhamnosus strain DSM33650 (about 3.0 ppm of 2,3-butanedione and less than 1.0 ppm of acetaldehyde). Acetaldehyde content was higher than diacetyl content in yogurt alternatives fermented with industrial starter cultures alone. The level of diacetyl in yogurt alternatives fermented with industrial starter cultures alone was lower than the yogurt alternatives fermented with industrial starter cultures in combination with the exemplary L. rhamnosus strain.

These results are supportive of the ability of strains of L. rhamnosus to decrease acetaldehyde content and increase diacetyl content in fermented soy-based yogurt alternatives, even when used in combination with industrial starter cultures.

### Example 3: Assessment of Cream Flavor in Fermented Plant-Based Yogurts

The ability to naturally produce and improve cream flavor in fermented plant-based yogurt alternatives through the use of bacteria, including industrial starter cultures containing Streptococcus thermophilus and Lactobacillus delbrueckii subsp. bulgaricus and exemplary Lacticaseibacillus rhamnosus strains including DSM33650, will be assessed.

Plant-based soy substrates will be inoculated, fermented, and stored generally as described in Example 2 above. Sensory analysis will be performed by a trained panel of sensory assessors who will evaluate the fermented plant-based yogurt alternatives via quantitative descriptive analysis (QDA) using a linear point scale. Statistical tests will be performed.

Sensory analysis of the fermented plant-based yogurt alternatives will be determined at different time points over the duration of storage.

These results will support a correlation between decreased levels of acetaldehyde, relative to diacetyl levels, and cream flavor in plant-based fermented yogurts. These results will further suggest that the inclusion of exemplary bacteria, and specifically exemplary L. rhamnosus strains, during fermentation can facilitate changes in volatile compounds to improve cream flavor in fermented plant-based yogurt alternatives.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

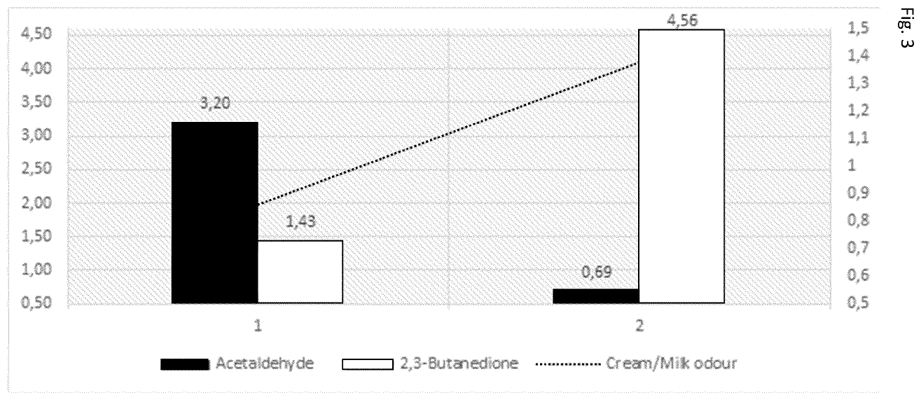

As shown in FIG. 3, after 14 days of storage, acetaldehyde content for soy yogurt alternative fermented with industrial starter culture alone is higher than soy yogurt alternative fermented with industrial starter culture in combination with L. rhamnosus strain DSM33650. After 14 days of storage, 2,3-butanedione content for soy yogurt alternative fermented with industrial starter culture alone is lower than soy yogurt alternative fermented with industrial starter culture in combination with L. rhamnosus strain DSM33650. Acetaldehyde content was higher than diacetyl content in soy yogurt alternative fermented with industrial starter culture alone. Acetaldehyde content was lower than diacetyl content in soy yogurt alternative fermented with industrial starter culture with the exemplary L. rhamnosus strain.

Cream/milk odour perceived by the sensory panel is higher in the soy yogurt alternative fermented with industrial starter culture in combination with L. rhamnosus strain DSM33650 than cream/milk odour perceived in the soy yogurt alternative fermented with industrial starter culture alone.

These results are supportive of the ability of strains of L. rhamnosus to decrease acetaldehyde content and increase diacetyl content in fermented soy-based yogurt alternatives, as well as to increase the perception of cream/milk odour.

These results illustrate that the inclusion of exemplary bacteria, and specifically exemplary L. rhamnosus strains, during fermentation facilitate changes in volatile compounds to improve cream flavor in fermented plant-based yogurt alternatives.

1. A method for producing a fermented plant-based composition having a cream flavor, the method comprising:
(a) fermenting a vegetal base inoculated with a starter culture to produce a fermented plant-based composition; and
(b) decreasing acetaldehyde content in the fermented plant-based composition.

2. The method of claim 1, comprising storing the fermented plant-based composition.

3. The method of claim 2, wherein:
(a) the acetaldehyde content is decreased in the fermented plant-based composition during storage to produce a ratio of acetaldehyde to diacetyl that is less than 0.7, 0.6, 0.5, 0.4, 0.3, 0.2, 0.1 or is 0; or
(b) the acetaldehyde content is decreased and a diacetyl content is increased during storage to produce a ratio of acetaldehyde to diacetyl that is less than 0.7, 0.6, 0.5, 0.4, 0.3, 0.2, 0.1 or is 0.

4. The method of claim 1, wherein the starter culture comprises one or more Lacticaseibacillus rhamnosus strains, wherein the one or more Lacticaseibacillus rhamnosus strains comprise or consist of:
(a) the Lacticaseibacillus rhamnosus strain deposited under accession number DSM33650 at the DSMZ or a mutant thereof;
(b) the Lacticaseibacillus rhamnosus strain deposited under accession number DSM22876 at the DSMZ or a mutant thereof; and/or
(c) the Lacticaseibacillus rhamnosus strain deposited under accession number DSM33850 at the DSMZ or a mutant thereof.

5. The method of claim 1, wherein the starter culture comprises one or more Streptococcus thermophilus strains, wherein the one or more Streptococcus thermophilus strains comprise or consist of the Streptococcus thermophilus strain deposited under accession number DSM33651 at the DSMZ or a mutant thereof.

6. The method of claim 1, wherein the starter culture comprises or consists of:
(a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof;
(b) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; or
(c) the Lacticaseibacillus rhamnosus strain deposited under accession number DSM33850 at the DSMZ or a mutant thereof and the Streptococcus thermophilus strain deposited under accession number DSM33651 at the DSMZ or a mutant thereof.

7. The method of claim 2, wherein the fermented plant-based composition:
(a) is stored at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or about 26° C.; and
(b) is stored for at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90 100, 110, or 120 days; or
(c) is stored for at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, 60, 70, 80, 90, 100, 110, or 120 days.

8. A composition comprising a Lacticaseibacillus rhamnosus strain, wherein the Lacticaseibacillus rhamnosus strain decreases acetaldehyde in a fermented plant-based composition to produce a cream flavor; wherein:
(a) the acetaldehyde content is decreased to produce a ratio of acetaldehyde to diacetyl that is less than 0.7, 0.6, 0.5, 0.4, 0.3, 0.2, 0.1 or is 0; or
(b) the acetaldehyde content is decreased and a diacetyl content is increased to produce a ratio of acetaldehyde to diacetyl that is less than 0.7, 0.6, 0.5, 0.4, 0.3, 0.2, 0.1 or is 0; and
wherein the composition is a starter culture or fermented plant-based food product.

9. (canceled)

10. The composition of claim 8, wherein the Lacticaseibacillus rhamnosus strain is:
(a) the Lacticaseibacillus rhamnosus strain deposited under accession number DSM33650 at the DSMZ or a mutant thereof;
(b) the Lacticaseibacillus rhamnosus strain deposited under accession number DSM22876 at the DSMZ or a mutant thereof; or
(c) the Lacticaseibacillus rhamnosus strain deposited under accession number DSM33850 at the DSMZ or a mutant thereof.

11. A fermented plant-based composition obtained by the method of claim 1, wherein the fermented plant-based composition is a fermented plant-based food product, a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.

12. (canceled)

13. A method of identifying bacteria capable of producing a fermented plant-based composition having cream flavor, the method comprising quantifying acetaldehyde content and diacetyl content in a fermented plant-based composition fermented in the presence of one or more bacteria, wherein the fermented plant-based composition is stored and the acetaldehyde content and diacetyl content are quantified during storage; and
if the quantified acetaldehyde content and diacetyl content produce a ratio of acetaldehyde content to diacetyl content that is less than 0.7, 0.6, 0.5, 0.4, 0.3, 0.2, 0.1 or is 0, the one or more bacteria are identified as capable of producing a fermented plant-based composition having cream flavor.

14. A composition comprising one or more bacteria identified as capable of producing a fermented plant-based composition having cream flavor according to the method of claim 13, wherein the composition is a starter culture or a fermented plant-based food product.

15. (canceled)
